SEQUENCE LISTING (6 sequences)

SEQ ID NO: 1 - protein, 1684 aa, Orthonairovirus sp.
MHISLMYAILCLQLCGLGETHGSHNETRHNKTDTMTTPGDNPSSEPPVSTALSITLDPSTVTPTTPASGLEGSGEVYTSPPITTGSLPLSETTPELPVTTGTDTLSAGDVDPSTQTAGGTSAPTVRTSLPNSPSTPSTPQDTHHPVRNLLSVTSPGPDETSTPSGTGKESSATSSPHPVSNRPPTPPATAQGPTENDSHNATEHPESLTQSATPGLMTSPTQIVHPQSATPITVQDTHPSPTNRSKRNLKMEIILTLSQGLKKYYGKILRLLQLTLEEDTEGLLEWCKRNLGLDCDDTFFQKRIEEFFITGEGHFNEVLQFRTPGTLSTTESTPAGLPTAEPFKSYFAKGFLSIDSGYYSAKCYSGTSNSGLQLINITRHSTRIVDTPGPKITNLKTINCINLKASIFKEHREVEINVLLPQVAVNLSNCHVVIKSHVCDYSLDIDGAVRLPHIYHEGVFIPGTYKIVIDKKNKLNDRCTLFTDCVIKGREVRKGQSVLRQYKTEIRIGKASTGSRRLLSEEPSDDCISRTQLLRTETAEIHGDNYGGPGDKITICNGSTIVDQRLGSELGCYTINRVRSFKLCENSATGKNCEIDSVPVKCRQGYCLRITQEGRGHVKLSRGSEVVLDACDTSCEIMIPKGTGDILVDCSGGQQHFLKDNLIDLGCPKIPLLGKMAIYICRMSNHPKTTMAFLFWFSFGYVITCILCKAIFYLLIIVGTLGKRLKQYRELKPQTCTICETTPVNAIDAEMHDLNCSYNICPYCASRLTSDGLARHVIQCPKRKEKVEETELYLNLERIPWVVRKLLQVSESTGVALKRSSWLIVLLVLFTVSLSPVQSAPIGQGKTIEAYRAREGYTSICLFVLGSILFIVSCLMKGLVDSVGNSFFPGLSICKTCSISSINGFEIESHKCYCSLFCCPYCRHCSTDKEIHKLHLSICKKRKKGSNVMLAVCKLMCFRATMEVSNRALFIRSIINTTFVLCILILAVCVVSTSAVEMENLPAGTWEREEDLTNFCHQECQVTETECLCPYEALVLRKPLFLDSTAKGMKNLLNSTSLETSLSIEAPWGAINVQSTYKPTVSTANIALSWSSVEHRGNKILVSGRSESIMKLEERTGISWDLGVEDASESKLLTVSVMDLSQMYSPVFEYLSGDRQVGEWPKATCTGDCPERCGCTSSTCLHKEWPHSRNWRCNPTWCWGVGTGCTCCGLDVKDLFTDYMFVKWKVEYIKTEAIVCVELTSQERQCSLIEAGTRFNLGPVTITLSEPRNIQQKLPPEIITLHPRIEEGFFDLMHVQKVLSASTVCKLQSCTHGVPGDLQVYHIGNLLKGDKVNGHLIHKIEPHFNTSWMSWDGCDLDYYCNMGDWPSCTYTGVTQHNHASFVNLLNIETDYTKNFHFHSKRVTAHGDTPQLDLKARPTYGAGEITVLVEVADMELHTKKIEISGLKFASLACTGCYACSSGISCKVRIHVDEPDELTVHVKSDDPDVVAASSSLMARKLEFGTDSTFKAFSAMPKTSLCFYIVEREHCKSCSEEDTKKCVNTKLEQPQSILIEHKGTIIGKQNSTCTAKASCWLESVKSFFYGLKNMLSGIFGNVFMGIFLFLAPFILLILFFMFGWRILFCFKCCRRTRGLFKYRHLKDDEETGYRRIIEKLNNKKGKNKLLDGERLADRRIAELFSTKTHIG

SEQ ID NO: 2 - protein, 23 aa, Artificial sequence
  tPA secretion signal sequence
MDAMKRGLCCVLLLCGAVFVSPS

SEQ ID NO: 3 - DNA, 36 bp, Artificial sequence
  deltaMUC forward primer
gatctccatcttcaggttgtggctgccgtgggtctc

SEQ ID NO: 4 - DNA, 36 bp, Artificial sequence
  deltaMUC reverse primer
gagacccacggcagccacaacctgaagatggagatc

SEQ ID NO: 5 - DNA, 36 bp, Artificial sequence
  deltaMUC deltaGP38 forward primer
atcgctgggctcctcgctgtggctgccgtgggtctc

SEQ ID NO: 6 - DNA, 36 bp, Artificial sequence
  deltaMUC deltaGP38 reverse primer
gagacccacggcagccacagcgaggagcccagcgat

(19) United States
(12) Patent Application Publication
(10) Pub. No.: US 2022/0062404 A1
(43) Pub. Date: Mar. 3, 2022

(54) GP38-TARGETING MONOCLONAL ANTIBODIES PROTECT ADULT MICE AGAINST LETHAL CRIMEAN-CONGO HEMORRHAGIC FEVER VIRUS INFECTION

(71) Applicants: Aura Rae GARRISON, Knoxville, MD (US); Charles Jason SHOEMAKER, Stephens City, VA (US); Joseph Walter GOLDEN, Hagerstown, MD (US)

(72) Inventors: Aura Rae GARRISON, Knoxville, MD (US); Charles Jason SHOEMAKER, Stephens City, VA (US); Joseph Walter GOLDEN, Hagerstown, MD (US)

(21) Appl. No.: 17/418,357

(22) PCT Filed: Jan. 7, 2020
(86) PCT No.: PCT/US2020/012621

§ 371 Date: Jun. 25, 2021

Related U.S. Application Data
(60) Provisional application No. 62/789,576, filed on Jan. 8, 2019.

(51) Int. Cl. A61K 39/12; C12N 7/00; A61P 31/14; C07K 16/10

(52) U.S. Cl. CPC A61K 39/12; C07K 16/10; A61P 31/14; C12N 7/00

(57) ABSTRACT

Crimean-Congo hemorrhagic fever virus (CCHFV) is an important human pathogen. Limited evidence suggests that antibodies can protect humans against lethal CCHFV disease, but the protective efficacy of antibodies has never been evaluated in adult animal models. Here adult mice were used to investigate the protection provided by glycoprotein-targeting neutralizing and non-neutralizing monoclonal antibodies (mAbs) against CCHFV infection. A single non-neutralizing antibody (mAb-13G8) was identified that protected adult type I interferon deficient mice >90% when treatment was initiated prior to virus exposure and >60% when administered after virus exposure. Neutralizing antibodies known to protect neonatal mice from lethal CCHFV infection, failed to confer protection regardless of IgG subclass. The target of mAb-13G8 was identified as GP38, one of multiple proteolytically-cleaved glycoproteins derived from the CCHFV glycoprotein precursor polyprotein. Robust protection required complement activity, but not Fc-receptor functionality. Consistently, it was found that GP38 previously identified as a secreted molecule also localizes to viral envelope and cellular plasma membranes. This study reveals GP38 as an important antibody target for CCHFV and lays the foundation to develop novel vaccines and immunotherapeutic against CCHFV in human.

### CROSS REFERENCE TO RELATED APPLICATIONS

This application claims the benefit of U.S. Provisional Application Ser. No. 62/789,576, filed Jan. 8, 2019, the contents of which are herein incorporated by reference in their entirety.

### STATEMENT AS TO RIGHTS OR INVENTIONS MADE UNDER FEDERALLY SPONSORED RESEARCH AND DEVELOPMENT

This invention was made with government support from the Medical Research Institute of Infectious Diseases, a subordinate organization of the United States Army Medical Research and Material Command. The United States government has certain rights in the invention.

### REFERENCE TO SEQUENCE LISTING SUBMITTED ELECTRONICALLY

The official copy of the sequence listing is submitted electronically via EFS-Web as an ASCII formatted sequence listing with a file named “3000050-003977_SEQLIST_ST25.txt”, created on Jan. 6, 2020 and having a size of 16,221 bytes and is filed concurrently with the specification. The sequence listing contained in this ASCII formatted document is part of the specification and is herein incorporated by reference in its entirety.

### BACKGROUND

Crimean-Congo hemorrhagic fever virus (CCHFV) is an enveloped virus in the Nairoviridae family (for a review see (1-3) that is spread in nature by ticks, primarily those of the genus Hyalomma. CCHFV has a tripartite, negative-sense RNA genome consisting of small (S), medium (M) and large (L) segments. While the S segment encodes the nucleocapsid protein (N) and the L segment encodes the RNA-dependent RNA polymerase, the M segment encodes the two structural glycoproteins (G_{N} and G_{C}) in addition to nonstructural glycoprotein products. CCHFV infects a large number of wild and domesticated mammalian species, including bovines and ovines, in addition to some avian species such as ostriches. Infections in these animals are predominantly asymptomatic, but can produce a prolonged (>5 days) viremia (4, 5). CCHFV infection in humans, caused through tick bites, exposure to infected animals, or nosocomial infections, can lead to an acute and potentially life-threatening disease termed Crimean-Congo hemorrhagic fever (CCHF) (2, 6, 7). Infection is characterized as a febrile illness with varying degrees of coagulopathy, liver injury, neurological manifestations, respiratory distress, lymphocytopenia and thrombocytopenia (3). The mortality rate ranges from 3-80% and this large range is theorized to depend on multiple factors including viral strain, route of exposure, speed of diagnosis, and access to emergency health care. There are currently no FDA approved drugs to treat CCHFV, although, there is conflicting evidence that ribavirin protects against lethal human disease (8, 9).

Passive antibody protection has been used in humans to protect against several viral hemorrhagic fever viruses, including New World arenaviruses and filoviruses (10, 11). Antibody-based therapies comprised of human survivor plasma have been used to treat CCHFV infected humans since the mid-20^{th} century (12-14). Two products, CCHF-Bulin and CCHF-Venin, both produced from plasma of convalescence patients, have been used in Bulgaria (14). These products are delivered either intramuscularly or intravenously, respectively. While some evidence suggests that these products can protect against CCHFV, there are a limited number of people treated and no controls used to verify the results. Human convalescent serum was used in Dubai during a small nosocomial outbreak and the five patients receiving the product survived, but two other patients were left untreated and succumbed to disease. These data suggest antibody therapies can protect against lethality (13). However, other studies have indicated antibody offers little protective efficacy (12). In general, passive immunotherapy against CCHFV in humans has produced mixed results with some studies demonstrating protective efficacy and others suggesting it is not protective. A major issue is the lack of statistical evidence these therapeutic options are efficacious owing to the limited number of cases where patients were treated. Overall, use of convalescent plasma, serum, or purified antibodies has been essentially abandoned due to safety issues regarding human convalescent products and the poorly defined nature of the product.

The CCHFV glycoproteins encoded by the M-segment are expressed as a precursor polyprotein that is proteolytically cleaved along the secretory pathway and eventually produces the two major glycoprotein components G_{N} and G_{C}, the latter of which is the only known target of neutralization (15-17). Prior to the production of the mature proteins, protease processing generates an intermediate molecule termed pre-G_{N} and G_{C}, then pre-G_{N} is further processed by protease to generate a G_{N} and other products such as GP38 that are secreted from cells and have unknown functions (15-19). A panel of murine monoclonal antibodies (mAbs) was produced against CCHFV strain IbAr10200 and several of these antibodies were identified as targeting the pre-G_{N} complex or the G_{C} protein. Many of the antibodies targeting G_{C} have neutralizing activity (20). Bertolotti-Ciarlet, A., et al demonstrated that both the non-neutralizing and neutralizing mAbs protect neonatal mice from lethality. Neonatal mice, however, do not recapitulate CCHFV disease making interpretation of these results difficult. The protective efficacy of glycoprotein-targeting mAbs has never been evaluated in adult animals.

### SUMMARY OF THE INVENTION

The present invention encompasses methods and compositions for use of non-neutralizing monoclonal antibodies to treat or prevent CCHFV infection. Encompassed herein are monoclonal antibodies that specifically bind to GP38 polypeptides, and methods of treating or preventing infection by CCHFV. Non-limiting embodiments of the invention include:

1. A non-neutralizing antibody for treatment against CCHFV infection, wherein the non-neutralizing antibody binds specifically to GP38.
2. The antibody of embodiment 1, wherein the antibody binds specifically to the amino acid sequence set forth in SEQ ID NO:1, or an amino acid sequence having at least 80% sequence identity to SEQ ID NO:1.
3. The antibody of embodiment 1 or embodiment 2, wherein the antibody comprises heavy chain CDR1, CDR2, and CDR3 having the same amino acid sequences as heavy chain CDR1, CDR2, and CDR3 of antibody mAb-13G8 and light chain CDR1, CDR2, and CDR3 having the same amino acid sequences as light chain CDR1, CDR2, and CDR3 of antibody mAb-13G8.
4. A fragment of the antibody of any of embodiments 1-3, which has specific binding activity to GP38, or variants or fragments thereof
5. A chimeric or a humanized antibody of any of embodiments 1-4.
6. A method of treating or preventing CCHFV infection in a subject wherein the subject is administered a composition comprising the antibody of any of embodiments 1-5.
7. The method of embodiment 6, wherein the antibody comprises heavy chain CDR1, CDR2, and CDR3 having the same amino acid sequences as heavy chain CDR1, CDR2, and CDR3 of antibody mAb-13G8 and light chain CDR1, CDR2, and CDR3 having the same amino acid sequences as light chain CDR1, CDR2, and CDR3 of antibody mAb-13G8.
8. The method of embodiment 6 or 7, wherein the antibody is administered to a subject after infection by CCHFV.
9. The method of embodiment 6 or 7, wherein the antibody is administered to a subject at risk of exposure to CCHFV.
10. A method for producing a chimeric antibody or humanized antibody of the antibody of embodiment 1, which comprises the steps of linking a DNA encoding a variable region of the antibody of embodiment 1 with a DNA encoding a constant region; inserting this into an expression vector; introducing the vector into a host; and producing the variable region and the constant region of the antibody.
11. The method of embodiment 10, wherein the antibody comprises heavy chain CDR1, CDR2, and CDR3 having the same amino acid sequences as heavy chain CDR1, CDR2, and CDR3 of antibody mAb-13G8 and light chain CDR1, CDR2, and CDR3 having the same amino acid sequences as light chain CDR1, CDR2, and CDR3 of antibody mAb-13G8.
12. The method of any of embodiments 6-11, wherein the subject is a mammal.
13. A humanized antibody for treating a CCHFV infection in a mammalian subject, wherein the antibody specifically binds to the amino acid sequence set forth in SEQ ID NO:1, or an amino acid sequence having at least 80% sequence identity to SEQ ID NO:1.
14. The humanized antibody of embodiment 13 that comprises a DNA encoding a variable region of the antibody of embodiment 1 and a DNA encoding a constant region.
15. The humanized antibody of embodiment 14, wherein the antibody comprises heavy chain CDR1, CDR2, and CDR3 having the same amino acid sequences as heavy chain CDR1, CDR2, and CDR3 of antibody mAb-13G8 and light chain CDR1, CDR2, and CDR3 having the same amino acid sequences as light chain CDR1, CDR2, and CDR3 of antibody mAb-13G8.
16. The humanized antibody of embodiment 15, wherein at least three independent epitopes are present and associated with SEQ ID NO:1.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIG. 1 demonstrates that mAb-13G8 protects against lethal disease in IFNR^{-/-} mice. A. IFNR^{-/-} mice (n=10 per group) were infected with CCHFV strain IbAr10200 by the subcutaneous (SC) route. Mice were injected with mAb-13G8, mAb-8A1 or a combination of the two on day -1 (1 mg total mAb concentration) by the IP route. As a control, mice were treated with PBS. Survival and percent group weight change were recorded. Log-rank test; ****p<0.0001. B. IFNR^{-/-} mice (n=10 per group) were infected with virus as in panel A. Mice were treated with two doses of the mAb-13G8 (1 mg/dose) or PBS on days −1/+3, +1/+4 or +2/+5 and survival and weight monitored. Log-rank test; ****p<0.0001, **p=0.0030. C. IFNR^{-/-} mice (n=10 per group) were infected as in panel A. Mice were treated with two doses of mAb-13G8, mAb-11E7 or a combination of both (1 mg total mAb concentration/dose) and survival and weight monitored. A non-specific murine antibody was used as a control. Log-rank test; ****p<0.0001.

FIG. 2 shows histologic and in situ hybridization (ISH) analysis of CCHFV infected mice treated with mAb-13G8. A. Representative H&E staining of livers and spleens from mice infected with CCHFV strain IbAr10200 and treated with mAb-13G8 or an isotype control antibody. Uninfected mice, treated with mAb-13G8, served as a negative control. Isotype-treated mice show a progressive liver and spleen deterioration which is not present in infected or uninfected mice treated with mAb-13G8. B. Representative ISH staining showing the presence of CCHFV RNA (red) in the liver and spleen of mice taken on day 4 post-virus exposure. Cells were counterstained with hematoxylin. C. Representative IHC stain for CCHFV N protein in liver and spleens of mice. Cells were counterstained with hematoxylin.

FIG. 3 shows the interaction of mAb-13G8 with the GP38 molecule. A. Schematic depicting CCHFV M-segment glycoprotein processing. B. The indicated mAbs were serially diluted tenfold (starting at 1:100) and incubated with purified G_{N}-ectodomain. H3C8 is an irrelevant murine mAb. Sera from a CCFHV infected human was used as a positive control along with a negative control human sample. C. Capture-ELISA was developed using mAb-13G8 or a negative control (mAb-QC03) as capture antibody and anti-M-segment polyclonal rabbit antisera as detection antibody. Two-way ANOVA; ****p<0.0001 D. 293T cells were transfected with constructs expressing the indicated proteins and protein expression analyzed by Western blot at 24 and 48h. G_{N} was detected using Anti-G_{N} 4093 (1:1000) and G_{C} was detected using mAb-11E7 (1:1000). Numbers on the left refer to molecular weight standard (kDa). E. Capture antibody for GP38 presence in the supernatant. For bait, 293T cells were transfected with plasmids expressing full-length strain IbAr10200 M-segment, ΔMucΔGP38, tpAGp3810200 or a negative control plasmid. Two-way ANOVA; **p<0.001, ***p<0.0001.

FIG. 4 demonstrates the heterologous protection of mice by mAb-13G8. A. Percent identical and percent divergence of CCHFV GP38 amino acid sequences from the indicated strains were determined using MegAlign Ves. 13.0.0 (DNAstar software). B. IFNR^{-/-} (n=10) mice were treated with two doses of the mAb-13G8 or an isotype control antibody on day −1/+3, and challenged SC with either IbAr10200 or Afg09-2990. Survival and percent weight change were monitored. Log-rank test; ****p<0.0001, ***p=0.0002. C. Representative ISH staining from livers and spleens harvested on day 4 from strain Afg09-2990 infected mice (n=3). ISH was conducted as in FIG. 2B. D. H&E of liver and spleen from Afg09-2990 infected mice taken on day 4 (n=3). Top left panel: focal area of mild inflammation (black arrow) and occasional Kupffer cell hypertrophy (arrow head). Middle left panel: a vessel is occluded by a fibrin thrombus (white arrow) and surrounded by an area of coagulative necrosis (circled). These mice were infected at the same time mice in FIG. 2. FIG. 5 shows that Fc-domains play a role in mAb-13G8-mediated protection. A. FcR^{-/-}, C3^{-/-} or B6:129 (wild-type) mice (n=8/group) were injected SC with two doses of the mAb-13G8 or an isotype control at D−1/+3 (1 mg/dose). IFN-I was blocked on day +1 using mAb-5A3 (1mg/m1) injected introperitoneally (IP). Mice were challenged with 100 plaque forming units (PFU) of CCHFV IbAr10200 by the IP route. Survival and weight were plotted. Log-rank test;****p<0.0001, ***=0.0004, *v0.0201. B. B6 mice (n=8) were treated with mAb-10E11, mAb-11E7 or isotype control on day −1/+3 (1 mg/dose) and infected as in panel A. Log-rank test;*p=0.0433.

FIG. 6 shows GP38 localization to the viral envelope and plasma membrane. A. CCHF virus-like particles (VLPs) or Venezuelan equine encephalitis virus (VEE) VLPs were incubated with the indicated antibodies and then stained with an anti-mouse secondary antibody conjugated to 10 nm gold particles. Samples were then examined by EM for the presence of GP38 or G_{C}. B. 293T cells were transfected with full-length M-segment (M-seg), AMAGP38 or an irrelevant protein (arenavirus GPC). To detect surface GP38 and G_{C}, non-permeabilized cells were incubated with mAb-13G8 or mAb-11E7, respectively. Cells were then stained with an anti-mouse secondary antibody conjugated with Alexafluor-488 and analyzed. 10,000 cells were counted per sample and data plotted with FlowJo software. C. Vero E6 cells were transfected with the indicated plasmids and incubated with MAb-10E11, then stained with an anti-mouse secondary antibody conjugated with Alexafluor-488 and analyzed by confocal microscopy.

FIG. 7 depicts the characterization of mAb and human convalescent sera interactions with the GP38 molecule. A. GP38his was plated in the wells of a 96-well plate (500 ng/well). The indicated mAbs were serially diluted tenfold (from 1:100) and incubated with purified protein. Endpoint titers were calculated as described in the materials and methods. Data was plotted as a mean titer for each group +/− standard deviation. *mAb-6B12 was run in a separate assay. B. Sera from CCHFV human survivors or a negative control sample were serially diluted in a purified GP38, G_{N}, or N protein ELISA. C. Epitope grouping of GP38-reactive mAbs based on competitive ability. Antibodies were grouped based on binding permissivity relative to their competing partner. Colors represent degree of permissivity with green denoting low competition (≥76), yellow weak competition (51-75), orange intermediate competition (26-51), and red high competition (≤25). D. Schematic depicting putative GP38 antibody competition groups using antibody from panel A.

FIG. 8 shows GP38 amino acid differences between CCHFV strains IbAr10200 and Afg09-2990. CCHFV GP38 amino acid differences from the indicated strains were determined using MegAlign Ves. 13.0.0 (DNAstar software). Only amino acid differences are shown on the Af09-2990 sequence.

FIG. 9 shows the combined ISH analysis of CCHFV strain IbAr10200 and Afg09-2990 infected mice treated with mAb-13G8. CCHFV RNA ISH analysis was performed as in FIG. 2C and FIG. 4C. Cells were counterstained with hematoxylin.

FIG. 10 shows the combined H&E analysis of CCHFV strain IbAr10200 and Afg09-2990 infected mice treated with mAb-13G8. H&E staining from livers of CCHFV infected mice was performed as in FIG. 2D and FIG. 4D. With the exception of the uninfected controls, sections in this figure are from different animals from the figures in the main manuscript. Box shows the approximant area of enlargement. Arrows point to inflammation and hepatocellular necrosis. Arrowhead notes hepatocytes exhibit lipid-type degeneration.

FIG. 11 shows the combined H&E analysis of CCHFV strain IbAr10200 and Afg09-2990 infected mice treated with mAb-13G8. H&E staining from spleens of CCHFV infected mice was performed as in FIG. 2D and FIG. 4D. With the exception of the uninfected controls, sections in this figure are from different animals from the figures in the main manuscript. Box shows the approximant area of enlargement. Arrows point to multifocal areas of inflammation.

FIG. 12 shows CCHFV lethality in IFN-I blocked BALB/c and BL6 mice. A. BL6 or BALB/c mice (n=8 per group) were infected with CCHFV strain Afg09-2990 by the IP route. On day +1 mice were treated with PBS (NEG), 0.5 or 2.5 mg of mAB-5A3 to block IFN-I signaling. Survival and percent group weight change were recorded.

FIG. 13 depicts antibody binding to GP38. A. To determine if these antibodies bound GP38, the coding region was fused with a domain of a poxvirus protein that binds to cell surfaces. The resultant construct termed CBD-GP38 was transfected into 293T cells and the interaction of various antibodies with these cells or cells transfected with an arenavirus GP1-CBD molecule (1) were evaluated by flow cytometry. mAb-13G8 and mAb-10E11 both interacted with CBD-GP38 on cell surfaces, but antibodies GC-targeting antibodies mAb-8A1 and mAb-11E7 did not. No antibody bound the negative control cells. B. Antibody competition between three CCHFV mAbs for binding on IbAr 10200 CCHF VLPs was assessed by analysis on the Octet platform. Presence of strong epitope competition (medium gray), weak competition (light gray), and no competition (dark gray) are indicated on table. Non-specific mAbs showed no evidence of competition (data not shown) failed to compete with any of the CCHF mAbs interrogated. C. Octet-based affinity analysis of CCHFV mAb interaction with recombinant GP38.

FIG. 14 shows that 10E11 does not protect against lethal disease in IFNR-/- mice. A. IFNR-/- mice (n=10 per group) were infected with 100 pfu CCHFV strain IbAr10200 by the SC route. Mice were injected with indicated antibodies by the IP route on day −1/+3 (1mg/dose). As a control, mice were treated with an isotype control antibody. Survival and percent group weight change were recorded.

FIG. 15 shows CCHF VLP neutralization by select mAbs with and without complement. VLP neutralization (80% inhibition) was generated for each mAb as previously described (Aura 2017) both with and without 5% rodent complement. The reciprocal value of the mAb concentration corresponding to the VLPNeut80 was then calculated (e.g. mAb with a VLPNeut80 of 1 μg/ml would be reported as 1, 10 μg/ml as 0.1, etc).

FIG. 16 shows GP38 binding of mouse and rabbit sera. Sera was produced in mouse (A) or rabbit (B) by DNA vaccination. Mice were vaccinated as described in (2). Rabbits were vaccinated as described elsewhere herein. For a GP38 target, 293T cells were transfected CBP-GP38 as in FIG. 10. Transfected cells were incubated with pooled sera from vaccinated mice (1:100) or individually vaccinated rabbits (n=2) (1:100) and then incubated with a secondary anti-mouse or anti-rabbit antibody conjugated with Alexafluor488 (1:500). Cells were washed and examined by flow cytometry. Sera from unvaccinated animals was used as a control.

In the Summary above, in the Detailed Description, and the claims below, as well as the accompanying figures, reference is made to particular features of the invention. It is to be understood that the disclosure of the invention in this specification includes all possible combinations of such particular features. For example, where a particular feature is disclosed in the context of a particular embodiment or embodiment of the invention, or a particular claim, that feature can also be used, to the extent possible, in combination with and/or in the context of other particular embodiments and embodiments of the invention, and in the invention generally. For the purposes of explanation, numerous specific details are set forth in order to provide a thorough understanding of the present invention. It will be apparent, however, to one skilled in the art that the present invention may be practiced without these specific details.

### DETAILED DESCRIPTION

Immunotherapeutics are effective treatment options against human viral infections, including orthopoxviruses (30), Rabies virus (31), Ebola virus (32), and Junin virus (11). Historically these products were comprised of hyperimmune serum from survivors (or vaccinated individuals), but more recently there is a greater interest in the use of monoclonal antibodies or in polyclonal products generated in transgenic animals that expresses human antibody. Indeed, crude antibody-based therapeutics have been developed and used against CCHFV since it first emerged in the 1940s (12-14). An inherent problem, however, was the fact these products were poorly characterized, had undefined potency, and carry a questionable safety profile due to their derivation from human blood products. Limited human data suggests immunotherapeutics against CCHFV are effective, but have never gained widespread use. In general, the use of immunotherapeutics as a therapeutic option against CCHFV has never been fully explored. This is partly due to the historic lack of adult animal models, (mice and nonhuman primates), which has only recently been rectified, to study CCHFV disease and medical countermeasure (MCM) efficacy. Dowall, S. D., el al, demonstrated in adult mice that antibodies from mice vaccinated with a CCHFV MVA-based vaccine failed to protect, despite neutralizing antibody activity, suggesting other factors such as T-cells are critical correlates of protection (33). Prior to this study, the only animal model whereby antibodies have been shown to protect against CCHFV were neonatal mice (20).

That study tested the efficacy of a panel of murine mAbs targeting pre-G_{N} and G_{C}. They found that antibodies against both pre-G_{N} and G_{C} protected 2-3 day old mice suggesting that both neutralizing (G_{C} targeting) and G_{N} targeting antibodies have protective efficacy. Using several of the more protective antibodies identified in this panel, the present invention demonstrates that most fail to provide protection against lethal CCHFV infection in adult mice. In two adult mouse models no neutralizing antibody, regardless of IgG subclass, provided even a minor amount of protection (delayed mean time to death (MTD), limited weight loss), thus neonatal mice do not predict protective efficacy in adult animals.

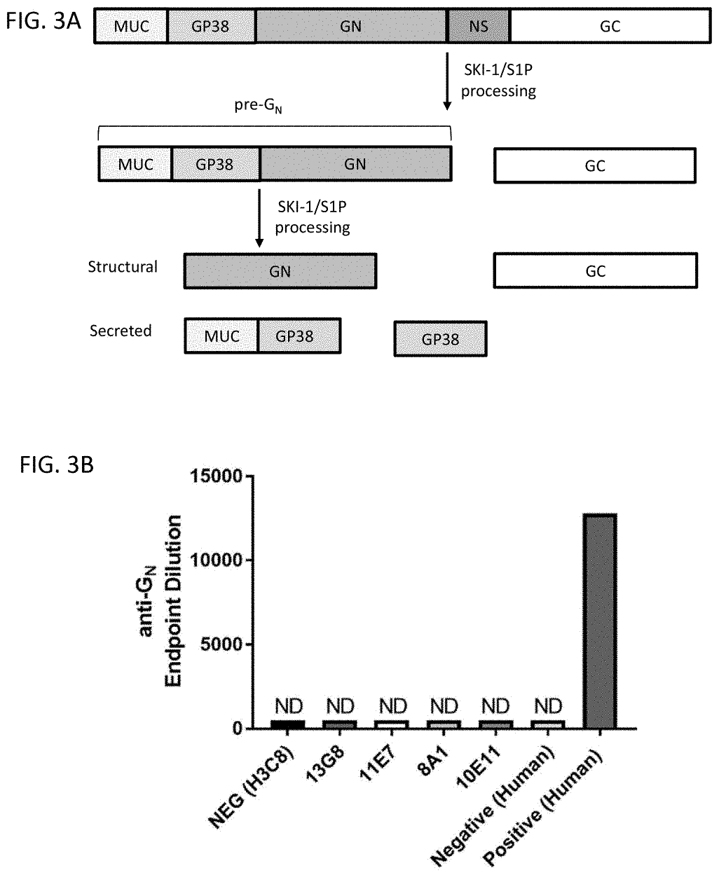

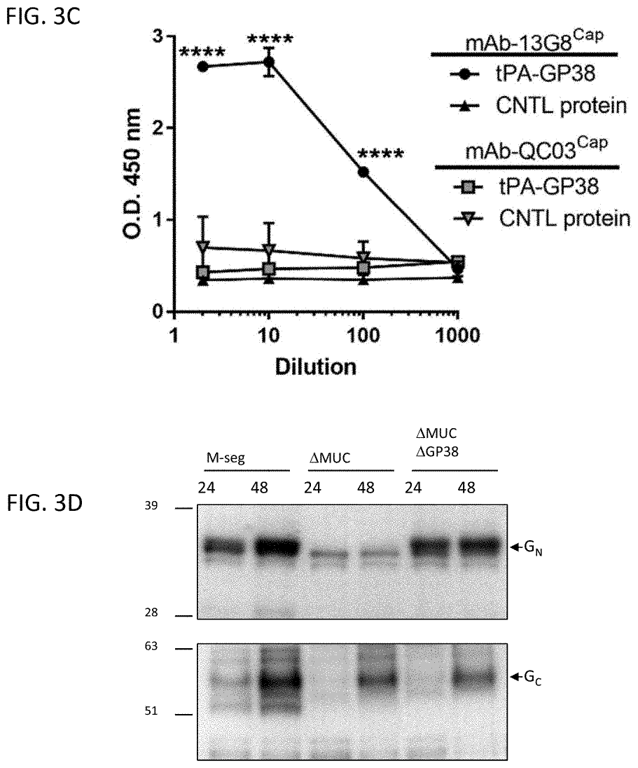

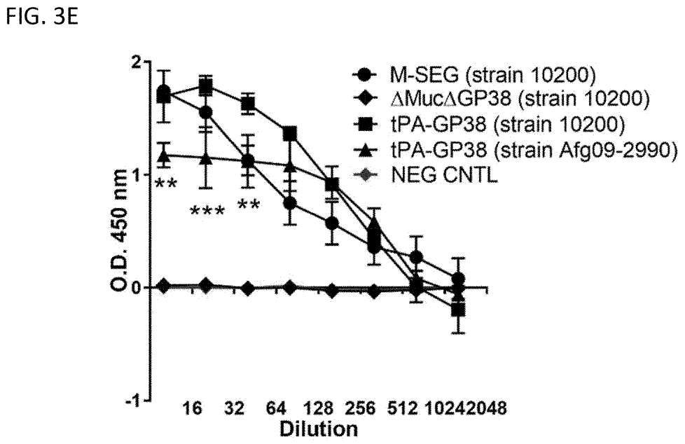

Prior to the present invention, the target of mAb-13G8 was identified as pre-G_{N}, a region of the unprocessed precursor glycoprotein encoded by the M-segment encompassing multiple domains including the mucin-like domain, GP38 and G_{N} ((17) and FIG. 3A). Based on the understanding that GP38 is a secreted molecule (17, 19), it was anticipated that protective efficacy would not require complement and/or Fc-receptor function and mAb-13G8 was blocking an unknown deleterious function(s) of a secreted viral toxin. The present invention unexpectedly demonstrates that complete protection requires functional complement activity. This finding is supported by the inability of an anti-GP38 IgG1 subclass of antibody, mAb-10E11, to protect mice from lethal infection, though mAb-10E11 did extend the MTD. Murine IgG1 antibodies poorly facilitate Fc-mediated effector functions. In neonatal mice, mAb-10E11, similar to mAb-13G8, was extremely protective and it is shown to compete with mAb-13G8 for binding on GP38. However, the results in neonatal mice failed to predict protection in adult mice, and mAb-10E11 does not bind GP38 with as high of efficacy as mAb-13G8. Nevertheless, the prospect that complement is required for protection is supported by the novel finding that GP38 is not only secreted, but also becomes localized to the viral envelope and cellular plasma membranes. Furthermore, the fact that mAb-13G8 inhibited spread of CCHFV to the liver after SC infection also supports a model whereby virus or virally-infected cells are being actively targeted for clearance. If mAb-13G8 only blocked the function of a viral toxin, it could be predicted that virus would traffic to a key tissue target, but virulence would be limited due to blockade of GP38 function. Thus, the present invention has demonstrated a novel and unprecedented mechanism(s) of CCHFV inhibition.

Protective non-neutralizing antibodies (nNAbs) have been reported for diverse groups of viruses including alphaviruses, HIV, and flavivirus (25-28). Passive protection elicited by the nNAbs often involves Fc-functionality, and several studies suggest that complement-dependent cytotoxicity (CDC) and/or ADCC play predominate roles (24). The nNAbs against flaviviruses target the non-structural protein 1 (NS1) and require antibody-dependent cellular cytotoxicity (ADCC) for protective efficacy (28). The NS1 molecule is similar to CCHFV GP38, as it is a secreted viral toxin that plays a role in influencing immune responses by activating TLR4, disrupting endothelial barrier function and manipulating complement (34).

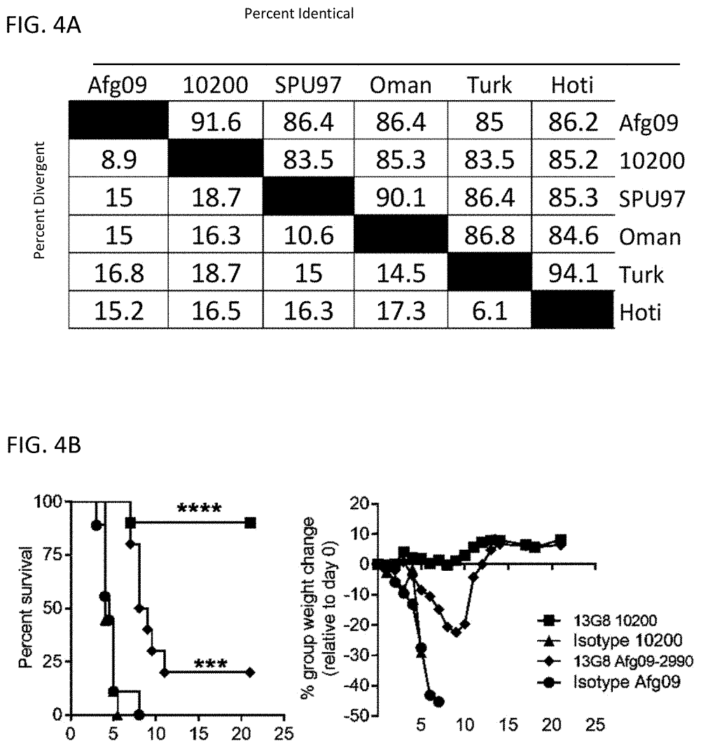

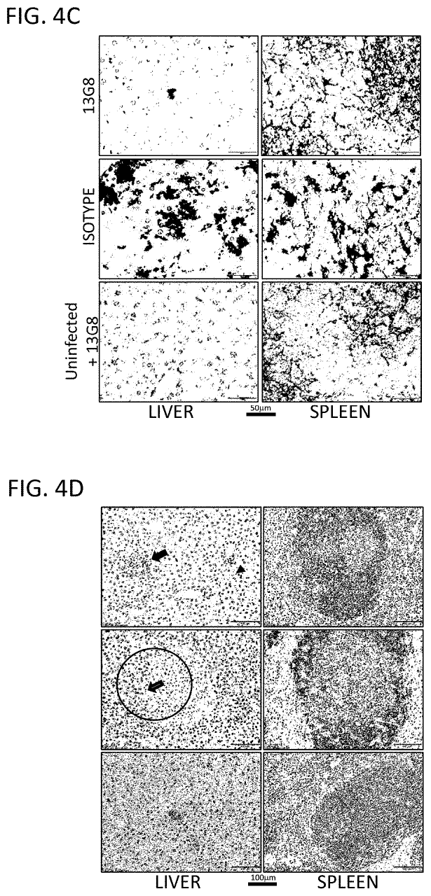

However, NS1 is also localized to plasma membranes in target cells where its role is poorly characterized (34). The present invention shows that GP38 is a secreted molecule that can localize to both the viral envelope and the plasma membrane and serve as a target of protective antibodies. CCHFV is genetically diverse, likely a result of the vast geographical regions where the virus circulates, which includes Africa, Asia, and Europe (3). This genetic diversity impacts virulence, and strains from different regions have widely varying degrees of lethality in humans (1). Due to this genetic diversity, CCHFV is divided into several linages (or clades) based on M and S-segment divergence (3). These differences can impact antigenicity of glycoproteins, including the interaction of neutralizing antibodies (35). GP38 similarly exhibits high diversity among lineages (FIG. 4A) and this impacted the cross-reactivity of mAb-13G8 (originally produced against strain IbAr10200) against GP38 derived from Afg09-2990. (FIG. 3E).

The pre-G_{N} mAbs reported by Bertolotti-Ciarlet et al, all bound GP38 in multiple assays and none of the antibodies bound to G_{N}. GP38 is also targeted in mice and rabbits vaccinated with plasmids expressing the full-length M-segment. Additionally, human sera taken from a small cohort (n=2) of African CCHFV survivors were universally positive for GP38, in addition to G_{N} and N. The present invention therefore suggests that GP38 is an important protective target of the anti-CCHFV humoral response. Poor IgG responses are nearly universal in fatal cases of CCHFV human disease. It is important to note that GP38 is not the only important target for CCHFV, as a DNA vaccine targeting G_{N}, G_{C}, and N, but excluding GP38 was 100% protective in adult mice (36). However, during active vaccination this protection could have been facilitated by T-cells.

G_{C}-targeting antibodies effectively neutralize CCHFV in cell culture (20, 35). However, the present invention indicates that this activity does not afford protection in two mouse models. Recent studies show that an M-segment DNA vaccine produced neutralizing antibody, but the levels of neutralizing antibody did not predict survival and did not correlate with protection, which was 60-70% (29). The present invention suggests that hematogenous dissemination of CCHFV in mice is not facilitated predominantly by free virus. Rather, virus may spread within targeted cells, such as neutrophils dendritic cells or macrophages. Alternatively, some viruses can cloak themselves in exosomes and avoid immune detection (37, 38). While not being bound to any particular theory or mechanism, CCHFV infection of certain cells in vivo may lead to the release of virus in a protected milieu, which is not recapitulated in contrived in vitro virus neutralizing assays.

CCHFV is endemic in Africa, Asia and Europe but is also emerging into new areas with the expansion of its vector, the Hyalomma ssp tick (3). Most recently autochthonous CCHFV infections were reported in Spain six years after CCHFV-positive ticks were identified in Southwestern Europe (39). These human infections included the index fatal case that resulted in fulminate hepatic failure and spread of the virus to a medical caregiver (6). This highlights the need for MCMs that can either prevent CCHFV infection or attenuate disease severity post-exposure. Because antibody provides instant immunity it is an attractive therapeutic option for limiting or preventing viral disease severity in a post-exposure setting. Thus, the present invention provides new methods and compositions for treating or preventing CCHFV, wherein the methods and compositions comprise use of a non-neutralizing antibody that specifically binds to GP38. A “non-neutralizing antibody” for the purposes herein is an antibody that binds specifically to virus particles, but does not neutralize infectivity. In various embodiments, the non-neutralizing antibody is mAb-13G8. As used in this invention, the term “GP38” comprises the full length GP38 protein set forth in SEQ ID NO:1. Furthermore, it will be understood by those of ordinary skill in the art, the amino acid sequence of GP38 can have naturally or artificial mutations (including but not limited to substitutions, deletions, and/or additions), not affecting its biological function. Therefore, in the present invention, the term “GP38” should include all such sequences and their natural or artificial variants. In various embodiments, the natural and artificial variants have at least 60% sequence identity to SEQ ID NO:1, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% sequence identity to SEQ ID NO:1.

As used in this invention, the term “antibody” refers to immunoglobulin proteins, which typically composed of two pairs of polypeptide chains (each pair has a “light” (L) chain and a “heavy” (H) chain). The light chains are classified as kappa and lamda light chains. The heavy chains are classified as mu, delta, gamma, alpha, or epsilon, and respectively, define isotype antibodies as IgM, IgD, IgG, IgA and IgE. In light chains and heavy chains, variable regions and constant regions are connected by a “J” region consisting of about 12 or more amino acids. The heavy chain also contains a “D” region with about 3 or more amino acids. Each heavy chain contains a variable region (V_{H}) and a constant region (C_{H}), which consists of 3 domains (C_{H1}, C_{H2}, and C_{H3}). Each light chain contains a variable region (V_{L}) and a constant region (C_{L}), which consists of one domain C_{L}. The constant region can mediate the binding of immune globulin to host tissues or factors, including various cells in the immune system (e.g., effector cells) and the complement component 1q (C1q) of the classical complement system. V_{H} and V_{L} can also be subdivided into regions with high variability (called complementarity determining region (CDR)), which are separated by relatively conservative regions called framework regions (FR). From the amino terminus to the carboxyl terminus, each V_{H} and V_{L} is composed of 3 CDRs and 4 FRs, in the order of FR1, CDR1, FR2, CDR2, FR3, CDR3, and FR4. The variable regions (V_{H} and V_{L}) of the heavy chain and light chain form the antibody binding site. Distribution of amino acids to the regions or domains follow the definitions by Kabat in Sequences of Proteins of Immunological Interest (National Institutes of Health Bethesda, Md. (1987 and 1991)), or Chothia & Lesk (1987) Mol. Biol., 196: 901-917; or Chothia et al. (1989) Nature, 342: 878-883. The term “antibody” is not restricted by any particular method of producing them. For example, it includes, in particular, recombinant antibodies, monoclonal antibodies, and polyclonal antibodies. Antibodies can be different isotypes, for example, IgG (such as IgG1, IgG2, IgG3, or IgG4 subtype), IgA1, IgA2, IgD, IgE, or IgM antibodies. In specific embodiments, the antibody is the monoclonal antibody mAb-13G8 (NR-40294, which is available through BEI Resources, Manassas, Va., USA).

The present invention further relates to chimeric antibodies, humanized antibodies, and human antibodies which can specifically recognize GP38, e.g., mAb-13G8. Chimeric antibodies are antibodies consisting of the variable regions of the heavy and light chains of a non-human mammal antibody such as a mouse antibody, and the constant regions of the heavy and light chains of a human antibody. Chimeric antibodies can be obtained, for example, by obtaining DNAs encoding the variable regions of the heavy and light chains of an antibody which can specifically recognize GP38, linking these DNAs with DNAs encoding the constant regions of the heavy and light chains of a human antibody, inserting them into an expression vector, and introducing the vector into a host, and producing the variable regions and constant regions of the antibody heavy and light chains. As the constant regions, constant regions derived from humans, mice, rats, rabbits, dogs, cats, cattle, horses, pigs, goats, rhesus monkeys, cynomolgus monkeys, chimpanzees, chickens, zebrafish, or such can be used. Modifications such as amino acid substitutions, deletions, and additions may be performed on the chimeric antibodies of the present invention to improve the stability of antibody production.

A humanized antibody is an antibody constructed by transferring the complementarity determining regions (CDRs) of an antibody derived from a non-human mammal such as mouse, to the complementarity determining regions of a human antibody. Humanized antibodies can be obtained, for example, by producing a DNA sequence designed to link DNAs encoding the CDRs of the heavy and light chains of an antibody which can specifically recognize GP38, and the human antibody framework regions (FR); inserting this into an expression vector; introducing the vector into a host; and expressing the protein encoded by the DNA. Modifications such as amino acid substitutions, deletions, and additions may be performed on the humanized antibodies of the present invention to, e.g., improve the stability of antibody production.

Human antibodies are antibodies prepared from mice which produce human antibodies. Human antibodies can be obtained, for example, by in vitro sensitization of human lymphocytes with desired antigens or cells expressing the desired antigens, and then fusing the sensitized lymphocytes with human myeloma cells. The human antibodies can also be obtained by immunizing transgenic animals carrying a complete repertoire of human antibody genes with desired antigens.

The compositions and methods herein further comprise antigen binding fragments. As used in this invention, the term “antigen binding fragments” refers to a polypeptide containing fragments of a full-length antibody, maintaining the ability to bind specifically to the same antigen (e.g., GP38), and/or to compete with the full length antibody to bind to the antigen, which is also called “the antigen binding portion.” Using conventional techniques known by those of ordinary skill in the art (such as recombinant DNA technology or enzymatic/chemical cleavage), an antigen binding fragment (such as the above described antibody fragments) may be obtained from a given antibody (e.g., mAb-13G8), and screened for specificity in the same manner as for the full antibody.

As used in this invention, the term “specific binding” refers to a non-random binding between two molecules, such as the interaction between the antibody and its target antigen. In some embodiments, a specific binding of an antibody to an antigen means an affinity (K_{D}), for example less than about 10⁻⁵ M, in particular, less than 10⁻⁶ M, 10⁻⁷ M, 10⁻⁸ M, 10⁻⁹M, 10⁻10 M, or less.

Furthermore, the present invention relates to an antibody which comprises CDR1, CDR2, and CDR3 having the same amino acid sequences as heavy chain CDR1, CDR2, and CDR3 of monoclonal antibody mAb-13G8, and CDR1, CDR2, and CDR3 having the same amino acid sequences as light chain CDR1, CDR2, and CDR3 of monoclonal antibody mAb13G8. Antibodies of the present invention may also have FR regions and constant regions.

All publications and patents mentioned herein are hereby incorporated by reference in their entirety as if each individual publication or patent was specifically and individually indicated to be incorporated by reference.

### EXPERIMENTAL EXAMPLES

### Example 1. Materials and Methods

### Ethics Statement

All animal studies were conducted in compliance with the Animal Welfare Act and other federal statutes and regulations relating to animals and experiments involving animals and adheres to principles state in the Guide for the Care and Use of Laboratory Animals, National Research Council (40). All animal experimental protocols were approved by a standing internal institutional animal care and use committee (IACUC). The facilities where this research was conducted are fully accredited by the Association for Assessment and Accreditation of Laboratory Animal Care International. Animals meeting criteria were humanly euthanized.

All data and human subjects research were previously de-identified and given a “research not involving human subjects” determination by the USAMRIID Office of Human Use and Ethics, OHU&E Log Number FY18-28.

### Viruses and Cells

Huh7 and SW13 cells were propagated in Dulbecco's Modified Eagles Medium with Earle's Salts (DMEM) (Corning) supplemented with 10% fetal bovine serum (FBS) (Gibco) 1% Penicillin/Streptomycin (Gibco), 1% Sodium Pyruvate (Sigma), 1% L-Glutamine (HyClone), and 1% HEPES (Gibco). Minimally passaged CCHFV strain Afg09-2990 (AF09) (41) or strain IbAr10200 (USAMRIID collection) were used for all experiments as indicated.

This virus was passaged three times in Huh7 cells. The virus was collected from clarified cell culture supernatants and stored at −80° C. All CCHFV work was handled in BSL-4 containment at USAMRIID.

### Anti-CCHFV and Isotype Antibodies

Anti-CCHFV murine mAbs are part of the USAMRIID hybridoma collection and have been described elsewhere (20). Antibody for murine challenges was purified in-house using the USAMRIID hybridoma facility. Murine isotype control antibodies for IgG2b and IgG1 were purchased from BioXcell. IgG2a isotype antibodies were purified in-house from a mAb-QC03 (Junin GP1) murine hybridoma.

### Mice

C57BL/6 (BL6), IFNR KO mice (B6.12952-Ifnar1tm1Agt/Mmjax), BL6;129 mice, and C3 knockout mice (B6;12954-C3tm1Crr/J) were obtained from The Jackson Laboratory. Fc receptor KO mice (C.129P2(B6)-Fcer1g^{tm1Rav} N12) were obtained from Taconic. Mice were all female and 6-15 weeks in age at the time of challenge.

### Passive Protection Experiments

Mice were challenged with 100 PFU of CCHFV strain IbAr10200 or Afg09-2990 by the subcutaneous (SC) (IFNR^{-/-}) or intraperitoneal (IP) (all other mice) route as indicated. Virus was diluted in a total volume of 0.2 ml PBS. All mice except IFNR^{-/-} were IP injected with 2.5 mg of anti-IFNR1 (mAb-5A3) (Leinco Technologies, Inc) diluted in PBS 24 h post-infection in a total volume of 0.4 ml. For antibody injections, as indicated mice were injected SC or IP with 1 mg/antibody/dose in a total volume of 0.2 ml diluted in PBS.

### Histology

Necropsy was performed on the liver and spleen. Tissues were immersed in 10% neutral buffered formalin for 30 days. Tissue were then trimmed and processed according to standard protocols (42). Histology sections were cut at 5-6 μM on a rotary microtome, mounted onto glass slides and stained with hematoxylin and eosin (H&E). Examination of the tissue was performed by a board-certified veterinary pathologist.

### In Situ Hybridization

CCHFV was detected in infected liver samples by ISH probes targeting IbAr10200 or Afg09-2990 M-segment of CCHFV as previously reported (21). Formalin-fixed paraffin embedded (FFPE) liver sections were deparaffinized and peroxidase blocked.

### IHC for Liver and Spleen

IHC was performed using EnVision IHC kit following the manufacture's protocol (Agilent). N protein was stained using the rabbit anti-CCHFV N protein (IBT Bioservices, 1:5000). Cells were counterstained with hematoxylin.

### Confocal Microscopy

Vero E6 cell monolayers were transfected in a 96-well Corning COC polymer plate with the indicated plasmids using Fugene 6. Transfected cells were incubated for 72 h in a 37° C. incubator with 5% CO₂. Cells were rinsed with PBS and fixed in 3.7% formalin for 10 m at room temperature. Fixed wells were subsequently rinsed against with PBS and blocked with 7.5% BSA in PBS (blocking buffer) overnight at 4° C. Samples were then incubated with mAb-10E11 (5 μg/μL) diluted 1:200 in blocking buffer overnight at 4° C. then rinsed three times with PBS. Following primary antibody incubation, PBS washed sections were incubated with Alexa Fluor 488-conjugated goat anti-mouse IgG antibody diluted (1:1000) in blocking buffer for 1 h at room temperature. Samples were then incubated with DAPI-PBS for 10 minutes at room temperature, rinsed in PBS and imaged

### Microscopy

Images were acquired using a Zeiss LSM 700, Zeiss LSM 880 confocal system or Olympus BX46. Images were processed using Zeiss Zen confocal software, CellSens software or ImageJ software.

### Cloning

All GP38 constructs were produced through de novo synthesis (GeneWiz; Germantown, Md.). tPA-GP38 strain IbAr10200 (NCBI Reference No. NC_005300) or strain Afg09-2990 (HM452306.1) were produced by the addition of the tPA secretion signal (MDAMKRGLCCVLLLCGAVFVSPS, SEQ ID NO:2). The cell binding version of GP38 was produced by adding the cell-binding domain (CBD) of the orthopoxvirus type I interferon binding protein (32) to the N-terminal region of GP38 from strain IbAr10200. Fusion of gene-products with the CBD allows the cell surface localization of the fusion product. Genes were cloned into the NotI and BglII sites of the pWRG7077 vector and verified by sequence analysis. The histidine tagged version of tPA-GP38 from strain IbAr10200, six histidine residues were added to the C-terminal domain of the protein by de novo synthesis and cloned into the HindIII and XhoI site of pBFksr-HCacc-MCS which contains a CMV promotor (Biofactora).

The codon optimized full length M-segment used was previously reported (29). The modified M-segments lacking the mucin and/or GP38 regions were produced by PCR. ΔMUC was produced using the forward primer 5′-GATCTCCATCTTCAGGTTGTGGCTGCCGTGGGTCT C-3′ having SEQ ID NO:3 and reverse primer 3′-GAGACCCACGGCAGCCACAACCTGAAGATGGAGATC-5′ having SEQ ID NO:4 which removed the mucin coding region in nucleotide regions 120-729. ΔMUCAGP38 was produced using the forward primer 5′-ATCGCTGGGCTCCTCGCTGTGGCTGCCGTGGGTCT C-3′ having SEQ ID NO:5 and reverse primer 3′-GAGACCCACGGCAGCCACAGCGAGGAGCCCAGCGAT-5′ having SEQ ID NO:6 primer sets which removed nucleotide regions 120-1545. Both ΔMUC and ΔMUCAGP38 constructs retained the signal sequence 1-117. All PCR reactions were performed using the Phusion polymersase (Invitrogen). Following PCR, fragments were digested with Notl and BglII, and ligated into the pWRG7077vector. Sequence analysis was used to verify that the changes had been successfully incorporated into the gene. Plasmids are listed in Table 1.

TABLE 1
Plasmid constructs

| Gene product | Plasmid name | Restriction sites |
|---|---|---|
| tPA-GP38 (IbAr10200) | pWRG/tPAGP3810200 | NotI/BglII |
| tPA-GP38 (Afg09-2990) | pWRG/tPAGP38AF09 | NotI/BglII |
| tPA-GP386xHIS (IbAr10200) | pBFksr-GP38his¹⁰²⁰⁰ | HindIII/XhoI |
| CBP-GP38 (IbAr10200) | pWRG/CBP-GP38¹⁰²⁰⁰ | NotI/BglII |
| M-segment(IbAr10200) | pWRG/M-segmentₒₚₜ | NotI/BglII |
| | 10200 | |
| ΔMuc M-segment(IbAr10200) | pWRG/M-segmentVer1 | NotI/BglII |
| ΔMurΔGP38 M-segment | pWRG/M-segmentVer2 | NotI/BglII |
| (IbAr10200) | | |

### GP38 Purification

Production of recombinant IbAr10200 GP38His was accomplished by transient transfection of HEK293Ts (ATCC) with the tPA-GP38His plasmid using Fugene 6 according to manufacturer's instructions. Supernatants were collected 48 and 72 h post-transfection (with media replacement after 48 h). Supernatants were clarified and mixed with protease inhibitors. Supernatants were then run over a Ni2+ HisTrap FF column (GE Healthcare) using an AKTA HPLC system. The column was then washed with 5 column volumes of binding buffer (500 mM NaCl, 10 mM Imidazole, 20 mM sodium phosphate, [pH 7.4]). Subsequently, captured GP38His was eluted off using Elution Buffer (binding buffer with addition of 500 mM Imidazole). Fractions with the highest A280 absorbance were then pooled and concentrated through a Centriprep 10 kDa filter device (Millipore). Final product protein concentration was determined by BCA (ThermoFisher).

### Flow Cytometry

293T cell monolayers were transfected in T25 flasks with the indicated plasmids using Fugene 6 (Promega). Transfected cells were incubated for 72 h in a 37° C. incubator with 5% CO2. Cells were detached with gentle tapping, were pelleted by centrifugation at 750× g and resuspended in 200 μL of FACS buffer (PBS, 5% FBS). Cells were incubated (1:100 dilution) with mAbs for 1 h at room temperature. Cells were then pelleted by centrifugation at 750× g and washed three times with FACS buffer. Cells were then incubated with anti-mouse Alexa fluor 488 (Invitrogen) (1:500) for 30 m at room temperature, washed three times and resuspended in 1 ml of FACS buffer. Flow cytometry was performed on a FACSCalibur flow cytometer (Becton Dickinson). Data were collected and analyzed using FlowJo software (Tree Star INC; Ashland, Oreg.). A total of 10,000 cells were analyzed for each sample using a live-gate.

### GP38 Capture ELISA

mAb-13G8 or mAb-QC03 (2.5 ug/ml) were diluted in 0.1 M carbonate buffer [pH 9.6], plated on high binding 96-well plate (Corning; Corning, N.Y.) and incubated overnight at 4° C. Plates were blocked for 1 h in blocking buffer [phosphate-buffered saline with 0.05% tween (PBST) containing 3% milk/3% goat sera] for 2 h @ 37° C. Plates were washed four times in PBST and incubated with supernatant from transfected 293T cells at the indicated dilution in blocking buffer for 2 h at 37° C. Plates were washed four times in PBST and incubated with an anti-M-segment antisera from DNA vaccinated rabbits (diluted 1:1200) in blocking buffer and incubated at 37° C. for 1 h. Plates were washed four times in PBST and incubated with anti-rabbit IgG conjugated to horseradish peroxidase (KPL) (1:1000) for 1 h at 37° C. Plates were washed again four times in PBST and 100 μL of Sureblue Reserve TMB microwell peroxidase 1-component (KPL) was added to each well. Reactions were stopped by adding 100 μL of TMB stop solution (KPL). The optical density (O.D.) at 450 nm was read on a TECAN microplate reader (TECAN Group Ltd.).

### GP38his ELISA

GP38his was diluted in 0.1 M carbonate buffer [pH 9.6] and plated in duplicate in the wells of a high binding 96-well plate (Corning). Plates were blocked for 1 h with PBST and 5% milk. Murine mAbs (ascites fluid) were serially diluted tenfold (starting from 1:100) in PBST containing 5% milk and E. coli lysate. Murine mAb dilutions were incubated with GP38his 1 h at 37° C. Plates were washed four times in PBST and incubated with an anti-mouse IgG conjugated to horseradish peroxidase (Sigma) (1:1000) for 1 hat 37° C. Plates were washed again four times in PBST and 100 μL of 2,2′-azinobis-3-ethylbenzthiazoline-6-sulfonic acid (ABTS) substrate (KPL) was added to each well. Reactions were stopped by adding 100 μL of ABTS stop solution of 5% (w/v) sodium dodecyl sulfate. The optical density (O.D.) at 405 nm was read on a TECAN microplate reader. End-point titers were determined as the highest dilution with an absorbance value greater than the mean absorbance value from negative control antibodies (mAb-11E7 and Anti-Junin virus GP1 mAb-QC03) plus three standard deviations. Mean titers were plotted using GraphPad Prism 7 software.

For analysis of human sera, high bind 96-well plates were coated with 500 ng/well of GP38, recombinant G_{N} (Native Antigen), or recombinant N (Native Antigen) O/N as described. The following morning, plates were blocked with Neptune blocking buffer (ImmunoChemistry Technologies) for 2 h at 37° C. Plates were then washed and probed with half-log dilutions (starting at 1:50) of sera from convalescent human survivors of CCHF in Neptune BB for 1 h at 37° C. After washing, anti-human IgG conjugated to horseradish peroxidase (Sigma) (1:1000) was added for 1 h at 37° C. Following a final wash, plates were treated with TMB substrate (SeraCare) at RT and reactions were arrested with TMB stop solution. The optical density (0.D.) at 450 nm was read on a TECAN ELISA plate reader, and mean titers were determined as stated above.

### DNA Vaccination

Anti-M-segment rabbit sera was produced by DNA vaccination of rabbits using a disposable syringe jet injection (DSJI) device (Pharmajet) as previously described (43). Rabbits were vaccinated with the full-length M-segment (pWRG7077/CCHFV-M-segmentₒₚₜ IbAr10200) at a concentration of 1 mg/dose of plasmid diluted in PBS in a total volume of 0.5 ml per injection. Rabbits were vaccinated three times at three week intervals.

### Western Blot

293T cells were transfected with plasmids encoding the IbAr10200 M-segment or ΔMUCAGP38-M using Fugene HD (Promega). Transfected 293T cells incubated at 37° C. for 24 or 48 h, after which cells were collected by low speed centrifugation and lysed in Tris lysis buffer (10 mM Tris [pH 7.5], 2.5 mM MgCl₂, 100 NaCl, 0.5% Triton X-100, 5 μg/μl of leupeptin [Sigma], 1 mM PMSF). 20 μL of sample were mixed with 4× protein sample buffer (0.125 M Tris [pH 8.0], 1% SDS, 0.01% [bromphenol blue, 10% sucrose) with the addition 10× reducing buffer. Protein samples then were analyzed by electrophoresis on SDS-10% polyacrylamide gels and transferred to PVDF membranes (Bio-Rad Laboratories). PVDF membranes were blocked for 2 h in phosphate-buffered saline (10 mM Tris [pH 8.0], 150 mM NaCl and 0.05% Tween) (PBST) containing 5% nonfat dry milk, rinsed with PBST, and incubated with Anti-G_{N} 4093 (1:1000). G_{C} was detected using mAb-11E7 (1:1000). Membranes were subsequently washed with PBST and incubated for 1 h with HRP-conjugated anti-rabbit (Anti-G_{N} 4093) (1:5000 in PBST) or anti-mouse (mAb-11E7) (1:10000 in PBST) (Amersham). Bound antibody was detected by treating the PVDF with SuperSignal West Femto chemiluminescent substrate detection reagents (Pierce) and photographed on a G-box (Syngene).

### VLP Production

CCHF and VEE VLPs were produced as previously described (29).

### Immunogold Staining and EM of VLPs

5 μl of CCHF or VEE VLPs were applied to formvar coated 200 mesh nickel grids and incubated 15-20 m. VLPs grids were then blocked with 4% normal goat serum (NGS) for 5 m, then wicked dry. Samples were then incubated for 20-30 m with either mAb-13G8 (1:500), mAb-11E7 (1:1000) or a negative control antibody H3C8 (1:1000). A control with buffer solution (without primary antibody) was prepared in parallel. Samples were then rinsed with buffer for 5 m. Secondary antibody (10 nm Gold; Goat anti-mouse; 1:25) was added to all samples and incubated for 15 m then washed 5 m with buffer then fixed with 1% glutaraldehyde for 1 m and rinsed in milliQ water. Samples were then negative stained for 1 m with 1% UA. Samples were subsequently imaged on Jeol 1011 TEM at various magnifications.

### Antibody Competition

A Tandem competition assay was used for binning monoclonal antibodies to CCHFV GP38 recombinant protein. Nickel charged tris-NTA (Ni-NTA) (Pall ForteBio part number 18-5101) sensors were loaded with rGP38his recombinant protein and equilibrated for 10 m in water, then 10 mM Nickel Chloride for 60 s and washed for 60 s in PBS. Sensors were then loaded with 10 μg/mL rGP38his recombinant protein by 5 m incubation in 1× Kinetics Buffer (ForteBio). Baseline readings were determined by equilibrating sensors for 60 s in 1× Kinetics Buffer. Purified mAbs against GP38 diluted with 1× Kinetics buffer to a concentration of 100 nM. Two null antibodies (EBOV-H3C8 and CCHFV-11E7) were also diluted and tested. One column of the Octet sample plate was used for seven mAbs and each assay included a no antibody control well. The eight sensors were incubated in the saturating antibody wells for 5 m before moving the sensors to a baseline well for 60 s in 1× Kinetics buffer. The sensors were regenerated by incubating them in solution of 10 mM glycine with a pH of 2.0 for 10 s followed by 10 s in PBS (pH 7.4). This regeneration cycle was performed three times before moving the sensors to a 1 m PBS wash. After washing, sensors were recharged with a 1 m incubation in a 10 mM Nickel Chloride solution. The sensors were then stored in water before using in additional assays. The data from the sensors was analyzed using the binning function of the Octet analysis software and competition groups assigned.

### Statistical Analysis

Weight loss was determined using one-way or two-way ANOVA with the Bonferroni correction. Survival statistics utilized the log-rank test. Significance levels were set at a p value less than 0.05. All analyzes were performed using GraphPad Prism 7 software.

### Example 2. Immunoprotection Against Lethal CCHFV Infection in IFNR-Deficient Mice

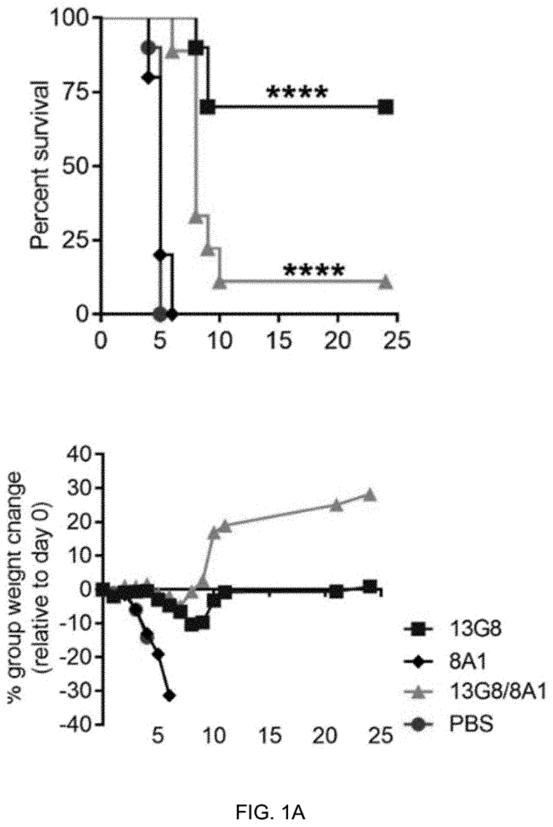

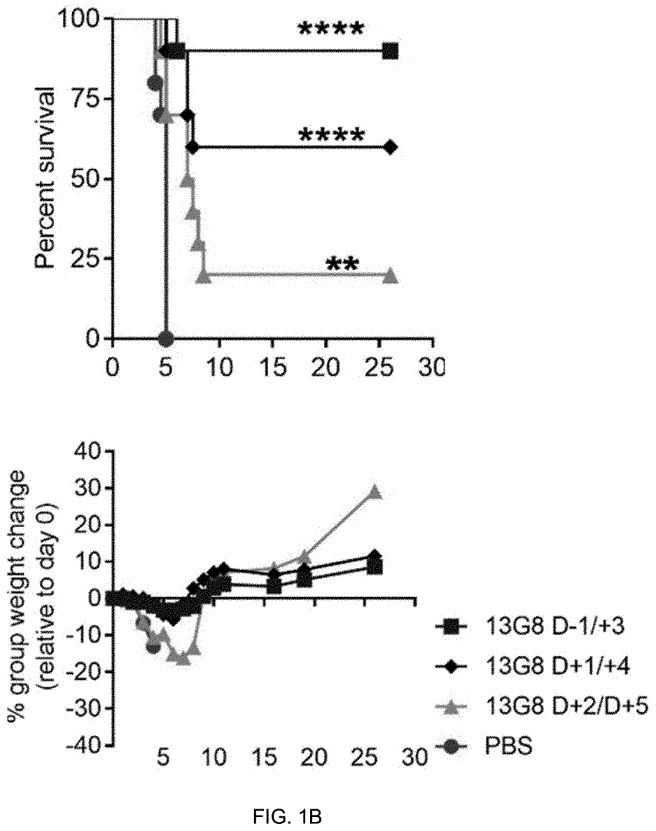

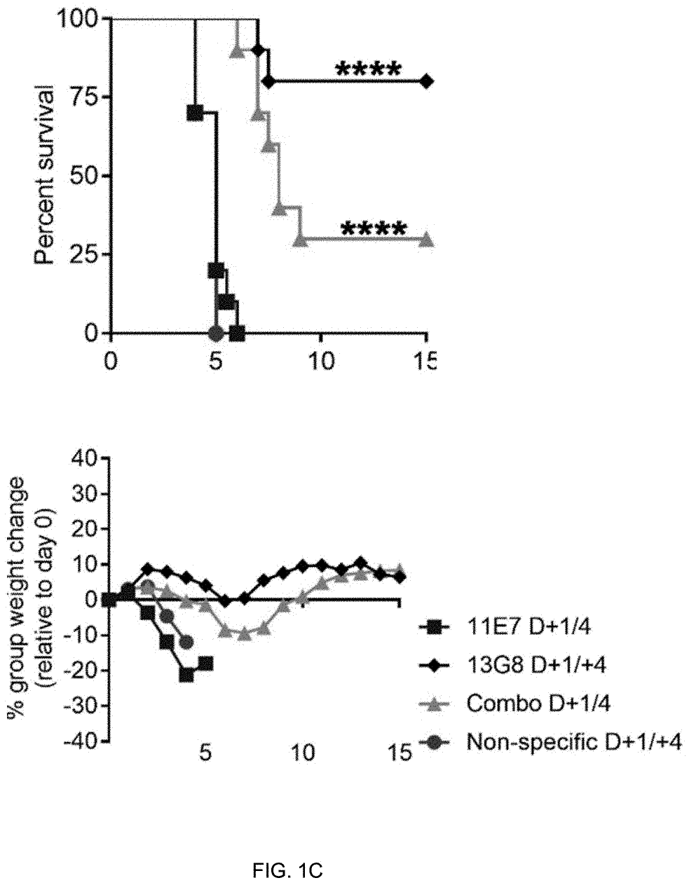

Groups of mice (n=10/group) were injected by the intraperitoneal (i.p) route with 1 mg total concentration of the indicated anti-CCHFV antibodies or PBS as a control, one day prior to virus exposure (FIG. 1A). Subsequently, mice were infected with 100 plaque forming units (PFU) of CCHFV strain IbAr10200 and weight and survival was monitored for 24 days. On day 3, negative control mice began to lose weight and all mice succumbed to infection by day 5. In contrast, mice treated with mAb-13G8 had significantly less weight loss that was also delayed compared to PBS treated mice. In this group, 6/10 mice survived and the mean time to death (MTD) was significantly delayed compared to the control group (log rank; p<0.0001). The neutralizing antibody mAb-8A1, which targets G_{C}, and robustly neutralizes virus in vitro (20) did not provide significant protection (log-rank; p=0.6004). A combination of mAb-13G8 and mAb-8A1 provided modest protection as measured by delayed weight loss and a significant delay in the MTD (log rank; p<0.0001). However, in this group only a single mouse survived to day 24.

Next it was determined if treating mice with multiple doses of mAb-13G8 would increase the protective efficacy. Additionally, mAb-13G8 was examined for protecting mice when treatment was initiated post-virus exposure. In this experiment, mice (n=10/group) were treated twice with mAb-13G8 either on days −1 and +3, days +1 and +4, or days +2 and +5 relative to infection (FIG. 1B). All PBS control treated mice succumbed to infection by day 5, after a period of weight loss. Mice treated with mAb-13G8 on day −1/+3 exhibited very little weight loss compared to control mice and 90% of the mice survived, which was significant (log rank; p<0.0001). Post-exposure treatment of mice on day +1/+4 regimen resulted in 60% survival (log rank; p<0.0001) and overall this group had reduced weight loss compared to PBS control treated mice. Treatment with mAb-13G8 on D+2/+5 conferred modest protection with 20% survival, which was significant (log-rank; p=0.0030) over control animals. However, weight loss in this group was not distinct from PBS-treated mice.

In an attempt to enhance the post virus exposure protective efficacy of mAb-13G8, mice were also treated with the neutralizing antibody mAb-11E7 on day +1 and +4. This mAb was included based on previous data suggesting both pre and post-exposure efficacy (20). Alone, mAb-11E7 failed to protect mice and the animals succumbed to infection by day 7 (FIG. 1C). Survival was not statistically distinct from isotype control treated animals (log rank; p=0.4243). Similar to the results above, 8/10 mice (80%) treated with mAb-13G8 alone survived which was significant (log rank; p<0.0001) and mice exhibited little weight loss. A combination of mAb-13G8 and mAb-11E7 (0.5 mg of each antibody/dose; 1 mg total/dose) failed to enhance protection over mAb-13G8 alone and only 3/10 mice survived challenge. However there was a significant delay in MTD (log rank; p>0.0001) compared to the control group. Mice treated with mAb-13G8 alone had a statistically significant survival advantage compared to the group receiving the combination of antibodies (log-rank; p=0.0353). Altogether, these findings indicate that mAb-13G8 protects adult mice against lethal infection, even when administered post-virus exposure. However, these findings also indicate that anti-G_{C} neutralizing antibodies (mAb-11E7 and the more potently neutralizing mAb-8A1) do not provide any protective benefit when given before or after virus challenge.

### Example 3. mAb-13G8 Blocks Virus Spread to the Liver and Spleen and Prevents Liver Pathology

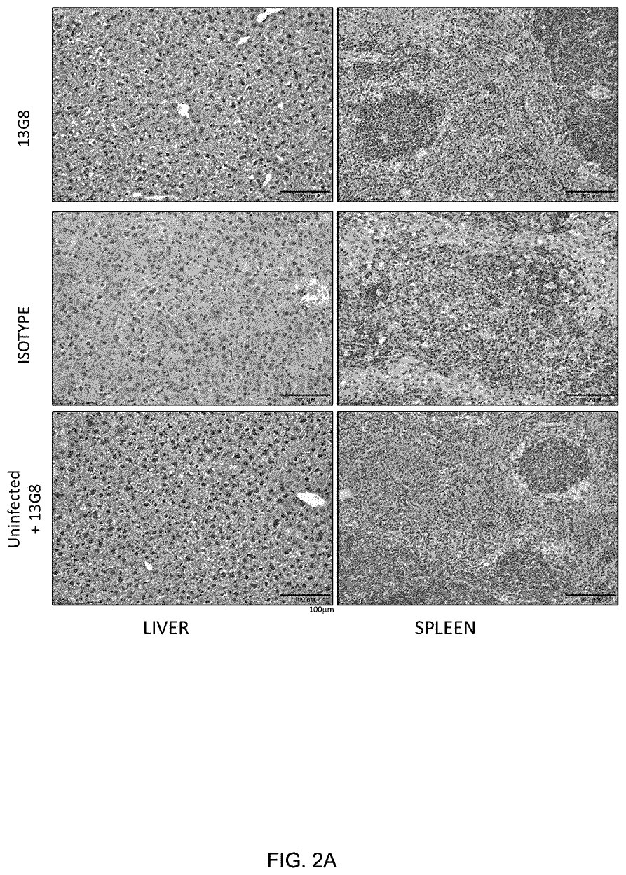

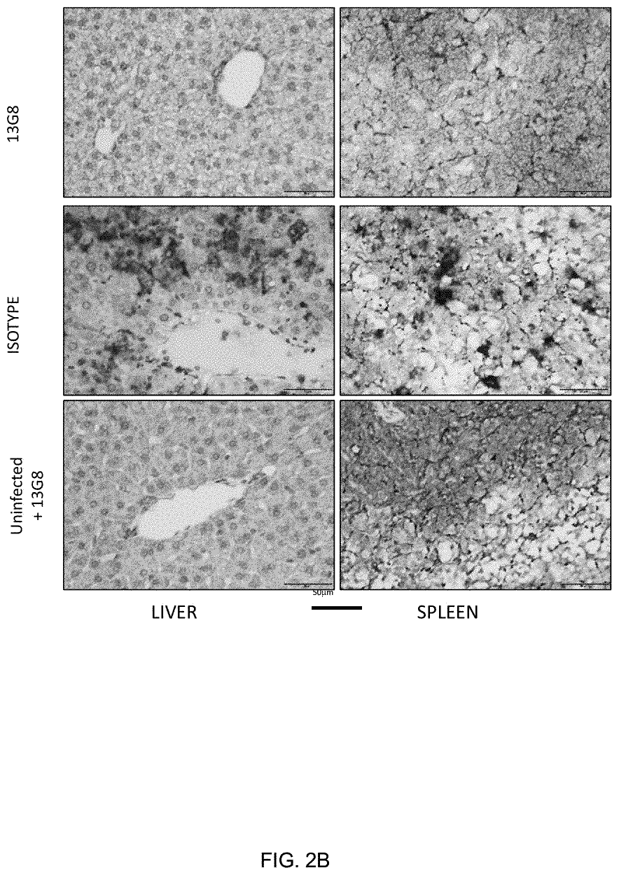

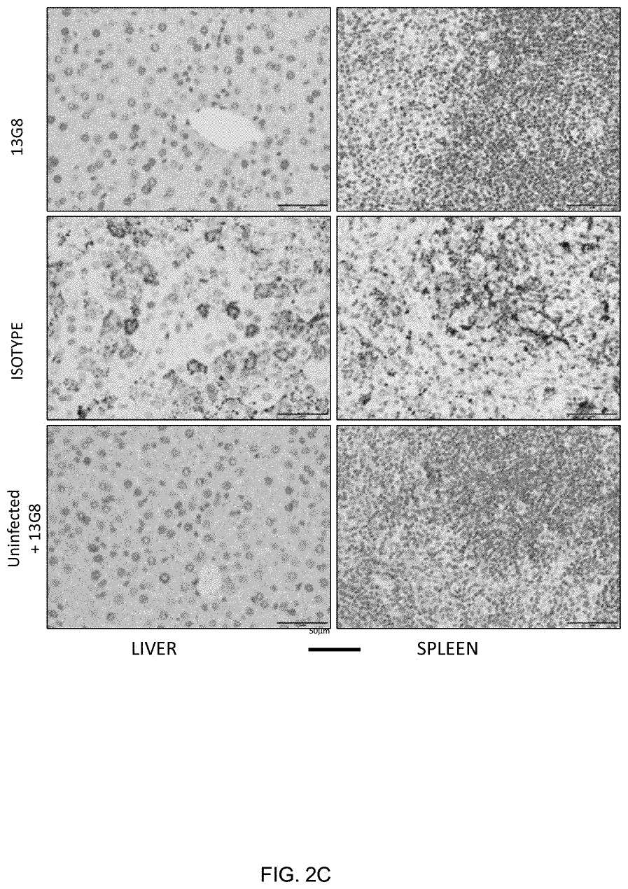

The primary targets of CCHFV pathogenesis and viral replication in mice are the liver and spleen (21, 22). The histopathological effects of CCHFV in these tissues were evaluated when mice (n=3 per group) were treated with mAb-13G8 or an isotype control antibody on day −1/+3 (FIG. 2 and Table 2).

TABLE 2

| | Group 1: | | | Group 2: | | | Group 3: | | | Group 4: | | | Group 5: | | |
|---|---|---|---|---|---|---|---|---|---|---|---|---|---|---|---|
| | IbAr10200 | | | Afg09-2990 | | | IbAr10200 | | | Afg09-2990 | | | UNINFECTED | | |
| Lesion^{a} | mAb-13G8 | | | mAb-13G8 | | | ISOTYPE | | | ISOTYPE | | | mAb-13G8 | | |
| Liver | | | | | | | | | | | | | | | |
| Inflammation | 1 | 1 | 1 | 1 | 2 | 1 | 5 | 3 | 5 | 3 | 3 | 5 | 1 | 1 | 1 |
| Kupffer cell | 0 | 0 | 0 | 0 | 1 | 0 | 2 | 2 | 2 | 2 | 0 | 0 | 0 | 0 | 0 |
| hypertrophy | | | | | | | | | | | | | | | |
| Hepatocellular | 0 | 0 | 0 | 0 | 0 | 0 | 5 | 2 | 3 | 3 | 3 | 3 | 0 | 0 | 0 |
| degen/necrosis | | | | | | | | | | | | | | | |
| Thrombus | 0 | 0 | 0 | 0 | 0 | 0 | 0 | 0 | 0 | 1 | 0 | 1 | 0 | 0 | 0 |
| Spleen | | | | | | | | | | | | | | | |
| Lymphoid | 0 | 0 | 0 | 0 | 1 | 1 | 0 | 0 | 0 | 0 | 0 | 0 | 0 | 0 | 0 |
| hyperplasia | | | | | | | | | | | | | | | |
| Inflammation | 0 | 0 | 0 | 1 | 0 | 0 | 0 | 2 | 0 | 0 | 0 | 0 | 0 | 0 | 0 |
| Lymphoid necrosis | 0 | 0 | 0 | 0 | 0 | 0 | 5 | 1 | 5 | 3 | 1 | 0 | 0 | 0 | 0 |

^{a}0 = lesion not present; 1 = minimal; 2 = mild; 3 = moderate; 4 = marked; 5 = severe

Tissues were examined on day 4, at the peak of disease in this model and 24 h prior to mice reaching euthanasia criteria (21, 22). Isotype control treated mice developed hepatic lesions with inflammation, hepatocellular necrosis with extensive hepatocellular degeneration and necrosis present in all animals (FIG. 2A). Additionally, periportal oval cell hyperplasia and Kupffer cell hypertrophy in the sinusoids was evident in isotype control treated, infected animals. Some hepatic vessels also contained fibrin deposits. In marked contrast, livers from mAb-13G8 treated, infected mice had no lesions and were indistinguishable from uninfected, mAb-13G8 treated mice. Spleens of isotype control treated, infected mice had moderate increases in neutrophils and histiocytes in the splenic red pulp (inflammation), and moderate lymphocytolysis in splenic white pulp were also observed (FIG. 2A). In contrast, splenic lesions were absent in infected, mAb-13G8 treated mice. Hepatic and splenic lesions in CCHFV-infected mice correlated with the presence of CCHFV RNA (FIG. 2B). In the spleen,

ISH signal was most prominent in the red pulp and in the marginal zone of the follicles, suggesting CCHFV was present within marginal zone macrophages and/or dendritic cells. In contrast, CCHFV was not detected in the liver or spleen of mAb-13G8 treated, infected mice. In addition to viral RNA, nucleocapsid protein (N) was also detected in the liver and spleen by immunohistochemistry (IHC) (FIG. 2C). These findings demonstrated that mAb-13G8 prevents damage to and limits viral replication in the liver and spleen when treatment is given on day −1/+3.

### Example 4. GP38 is the Target of mAb-13G8

Expression of the CCHFV M-segment produces a polyprotein precursor that is proteolytically cleaved into multiple glycoproteins, including G_{N} and G_{C} (FIG. 3A). During this cleavage event a precursor molecule called pre-G_{N}, consisting of the mucin domain, GP38 and G_{N} is also formed (16, 17). The target of mAb-13G8 is not well-characterized and initial studies indicated it and other antibodies, such as mAb-10E11, bound the pre-G_{N} complex. G_{N} was ruled out as a target of both mAb-13G8 and mAb-10E11 (FIG. 3B). Next, GP38 was evaluated as the target of mAb-13G8. To this end, a capture ELISA was developed using mAb-13G8 as a capture antibody and rabbit polyclonal CCHFV anti-M-segment antibody as a detection antibody (FIG. 3C). The rabbit polyclonal antibody was produced by DNA vaccination. As bait, a secreted molecule was designed by creating a construct consisting of the GP38 coding region with a tissue plasminogen activator secretion signal (tPA), termed tPA-GP38¹⁰²⁰⁰. Secreted tPA-GP38¹⁰²⁰⁰ in the supernatant of transfected 293T cells was readily detected when mAb-13G8 was used as the capture antibody, but negative control protein was not detected. These interactions were statistically significant (two way ANOVA: p<0.05) GP38 was also not detectable by a negative control capture antibody (mAb-QC03). In another experiment, M-segment variants were developed in which the mucin domain (ΔMuc) or the mucin and GP38 (ΔMucΔGP38) domains were removed. By western blot, expression of the G_{N} and G_{C} proteins from the ΔMucΔGP38 construct was similar to that from the wild-type M-segment at 48 h, however expression of these glycoproteins from the ΔMuc construct was greatly reduced (FIG. 3D). Because expression of G_{N} and G_{C} was not disrupted from the ΔMucΔGP38 construct, it was used to verify that mAb-13G8 binds to GP38 expressed from the M-segment precursor glycoprotein. 293T cells were transfected with full-length M-segment, the M-segment ΔMucΔGP38, tPA-GP38¹⁰²⁰⁰ and an irrelevant protein (Junin virus GP1). Supernatants from M-segment and tPA-GP38¹⁰²⁰⁰ transfected cells both interacted with mAb-13G8 (FIG. 3E). However, no interaction occurred with supernatant produced from ΔMucΔGP38 transfected cells or cells expressing a secreted negative control protein (JUNV GP1). A tPA-GP38 construct was also produced from CCHFV strain Afg09-2990 and termed tPA-GP38^{AF09}. In this assay, mAb-13G8 interacted with tPA-GP38^{AF09}, but to a lesser extent compared to tPA-GP38 from strain IbAr10200. This was statically significant at several dilutions (two-way ANOVA; p<0.05). These data indicate that the target of mAb-13G8 was the GP38 product of the M-segment.

### Example 5. Passive Protection by mAb-13G8 Against Heterologous CCHFV Strains is Limited

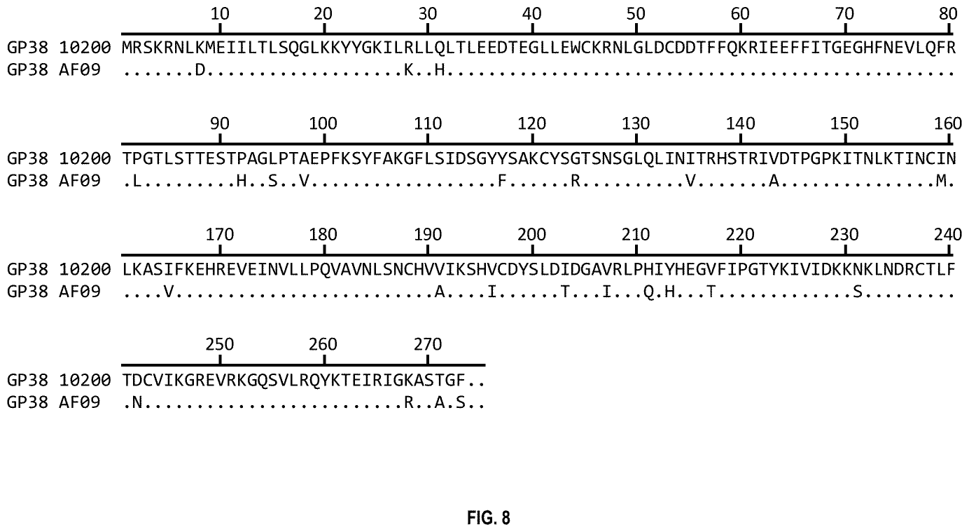

GP38 exhibits high heterogeneity (FIG. 4A). The cross-protective efficacy of mAb-13G8 was examined against a heterologous challenge using the CCHFV strain Afg09-2990 whose GP38 molecule has 26 amino acid differences compared to strain IbAr10200 (91.6% homology) (FIG. 8). Similar to strain IbAr10200, strain Afg09-2990 is lethal in IFN-I deficient mice and kinetics of infection are identical (21). Four groups of mice (n=12 per group) were treated with 1mg of mAb-13G8 or an isotype control on day −1/+3. Mice were infected with either strain IbAr10200 or strain Afg09-2990 and the protection was evaluated (FIG. 4B). As expected, mAb-13G8 treatment of strain IbAr10200 infected mice resulted in minimal weight loss and 90% survival, which was significant over isotype control mice which all succumbed to infection by day 5 (log rank; p<0.0001). In contrast, protection of mAb-13G8 against strain Afg09-2990 infected mice was diminutive and only resulted in 20% survival. However this was a significant increase compared to isotype control treated, Afg09-2990 infected mice (log rank; p=0.0002). Overall mAb-13G8 protected the homologous strain IbAr10200 infected mice significantly better compared to the heterologous strain Afg09-2990 infected mice (log rang; p=0.0028).

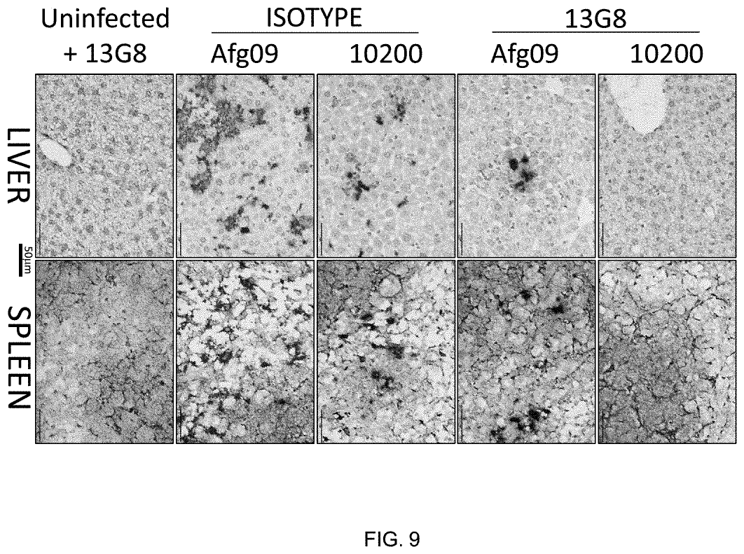

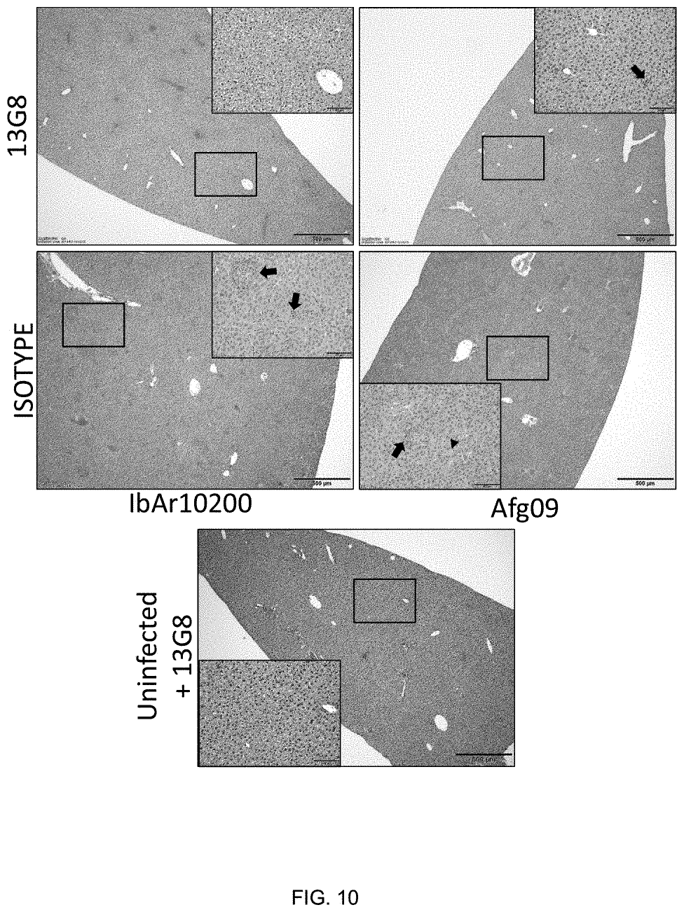

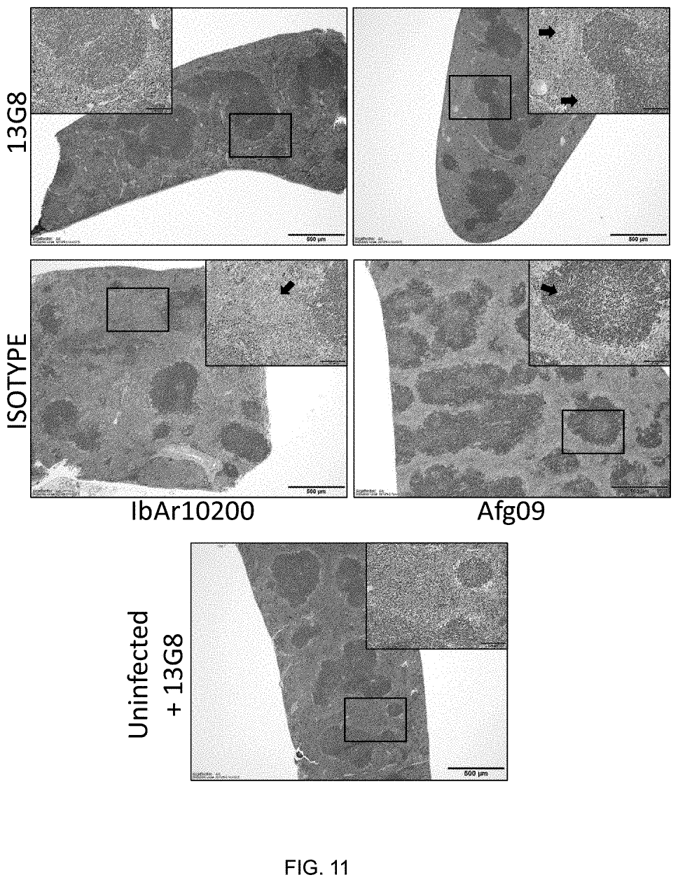

Histopathology and viral load in livers and spleens of strain Afg09-2990 infected mice (n=3 per group) were analyzed on day 4 (FIG. 4 and Table 2. Some viral mRNA was detected in both livers and spleens in all three strain Afg09-2990 infected mice, however staining was much more intense in isotype control treated mice (FIG. 4C and FIG. 9). This suggests mAb-13G8 was inhibiting viral replication to some extent at this time point. Despite this, the level of viral RNA in strain Afg09-2990 infected mouse livers and spleens was higher overall compared to those infected with strain IbAr10200 where staining was absent (FIG. 2C and FIG. 10 & FIG. 11). Liver lesions and inflammatory infiltrates in Afg09-2990 infected mice treated with mAb-13G8 were minimal to mild. However, in contrast to IbAr10200 infected mice, in some areas, neutrophils were the predominant inflammatory cell.

A single mouse in this group also exhibited rare Kupffer cell hypertrophy. Similar to the livers, splenic lesions were minimal. However, two animals exhibited minimal reactive lymphoid hyperplasia and one animal had a single focal area of minimal neutrophilic inflammation affecting splenic white pulp. In contrast all animals in the isotype control group exhibited microscopic hepatic lesions consistent with CCHF disease including moderate to severe inflammation, moderate hepatocellular degeneration and necrosis and Mild Kupffer cell hypertrophy. Lymphoid necrosis was also observed in two of the three Afg09-2990 infected isotype control mice. (FIG. 4D). These data indicated that mAb-13G8 can partially protect mice against heterologous CCHFV strains, but protection is markedly reduced compared to that against homologous virus.

### Example 6. mAb-13G8 Requires Complement Activity for Maximal Protection

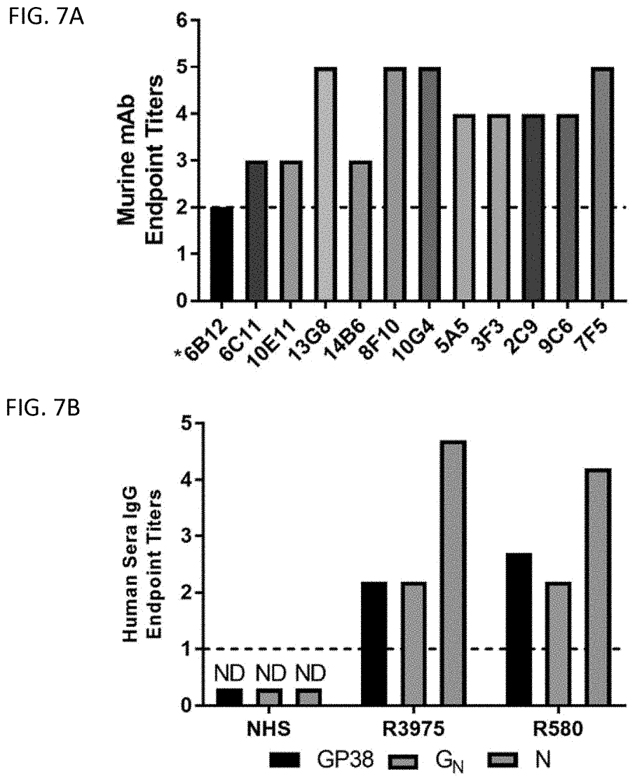

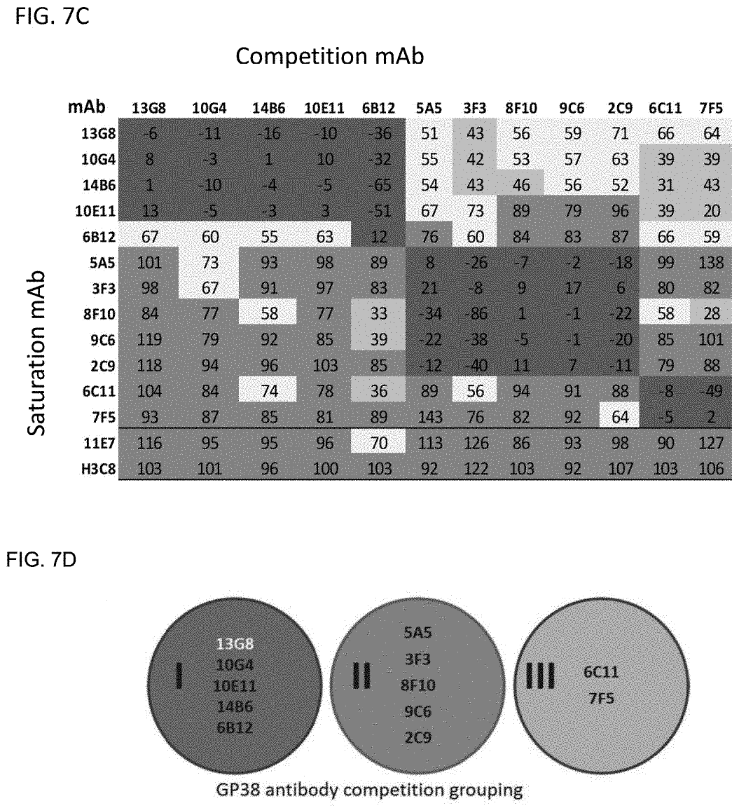

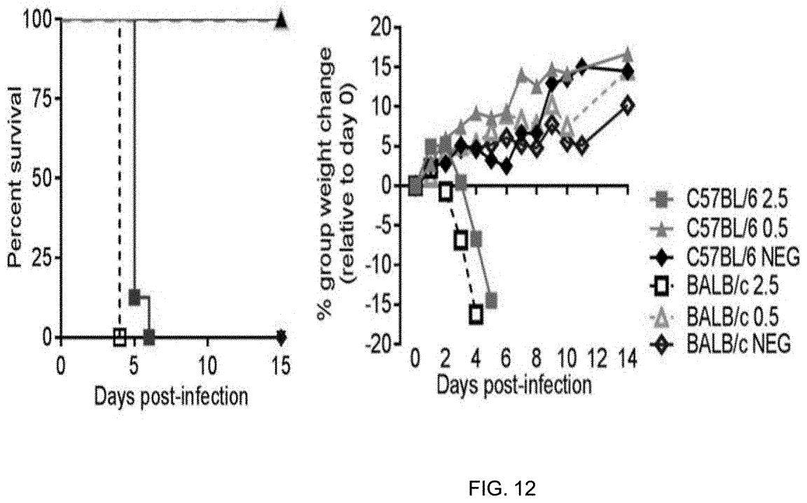

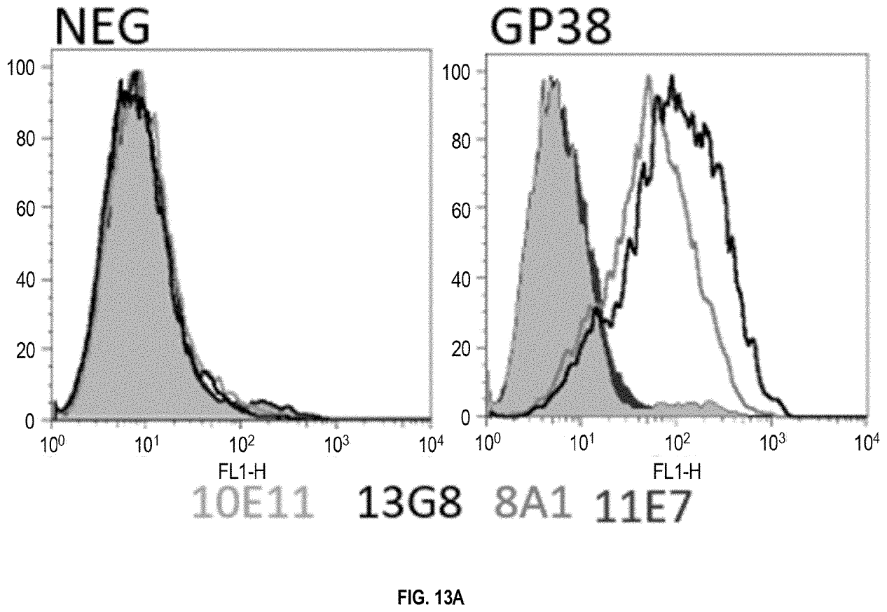

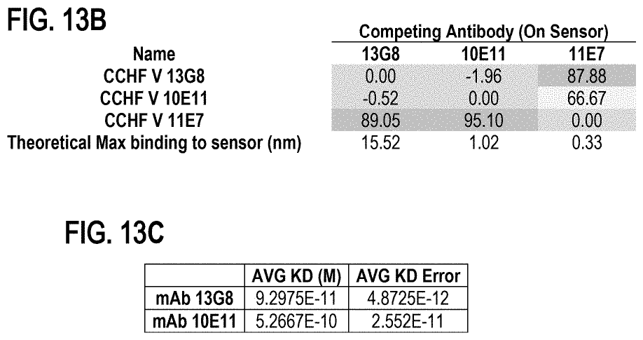

Non-neutralizing antibody can protect against viral infections through Fc-mediated processes such as antibody-mediated cytotoxicity (ADCC) or complement-mediated functions (24-28). The present invention evaluated if these processes were involved in mAb-13G8-mediated protection using Fc-receptor deficient (Fc^{-/-}) and C3 deficient (C3^{-/-}) mice as mAb-13G8 is an IgG2b isotype which can mediate these effector functions. These mice are unable to facilitate Fc-receptor function or complement-mediated activity, respectively. CCHFV only causes disease in mice when IFN-I signaling is blocked, however, a model system was developed using an antibody (mAb-5A3) to block IFN-I signaling which allows the exploration of CCHFV in essentially any transgenic model system (21, 29). In this system, kinetics of disease are identical to IFN-I receptor KO mice. For this experiment, mAb-13G8 and isotype control antibodies were injected subcutaneously (1mg/dose) on days −1 and +3 (FIG. 5A). Mice (n=8 per group) were infected intraperitoneally with CCHFV strain IbAr10200. On day +1 post-infection, mice were injected with mAb-5A3 which disrupted IFN-I activity. Wild-type and FcR^{-/-} mice were fully protected by mAb-13G8, but not by the isotype control antibody. Protection in FcR^{-/-} mice was significant (log rank; p<0.0001), as was protection in C57BL/B6:129 mice (log rank; p=0.0004). In contrast, mice lacking C3 (which were on a C57BL/B6:129 background) were poorly protected from CCHFV infection and mAb-13G8 only slightly delayed the mean time to death, which was significant (log rank; p=0.0201). Because FcR^{-/-} mice were on a BALB/c background, the lethality of CCHFV in this mouse strain was verified. Indeed, BALB/c mice were highly susceptible to CCHFV with lethality occurring 24 h prior to that of C57BL/6 mice, and similar to C57BL/BL6 mice, lethality required blockade of the IFN-I response (FIG. 12). These data support a role for complement activity, but not Fc-receptor activity in mAb-13G8 facilitated protection. Murine IgG1 antibodies are unable to efficiently capitalize on Fc-mediated effector functions. To confirm a requirement for Fc-domains in complement-mediated protection, an IgG1 subclass antibody that bound GP38 was identified (FIGS. 7A, 13A) and also protected neonatal mice from CCHFV (20). This antibody, mAb-10E11, also competed with mAb-13G8 for binding (FIG. 13B), but with an apparent lower affinity based on KD values (FIG. 13C). The ability for this antibody to protect adult mice from lethal infection was evaluated. Mice (n=8 per group) were injected subcutaneously (SC) (1mg/dose) on day −1/+3 with mAb-10E11 (anti-GP38), the neutralizing antibody mAb-11E7 (anti-G_{C}) or an isotype control antibody (FIG. 5B). Mice were infected IP with CCHFV strain IbAr10200 and IFN-I activity was blocked on day +1 using mAb-5A3. Neither mAb-11E7, mAb -10E11, nor the control antibody protected animals from weight loss or lethality. All mAb-10E11 and mAb-11E7 treated mice succumbed to disease by day 6. A single control animal survived challenge. There was a statistically significant difference between the isotype control mice and mice treated with mAb-10E11 in the MTD (log-rank: p=0.0433), suggesting a slight protective advantage. Similar results were produced in IFNR^{-/-} mice mAb-10E11 (FIG. 14). These results demonstrated that an IgG1 antibody targeting GP38, which competes against mAb-13G8, does not confer robust protection to infected mice.

### Example 7. GP38 Localizes to the Viral Envelope and Plasma Membrane.

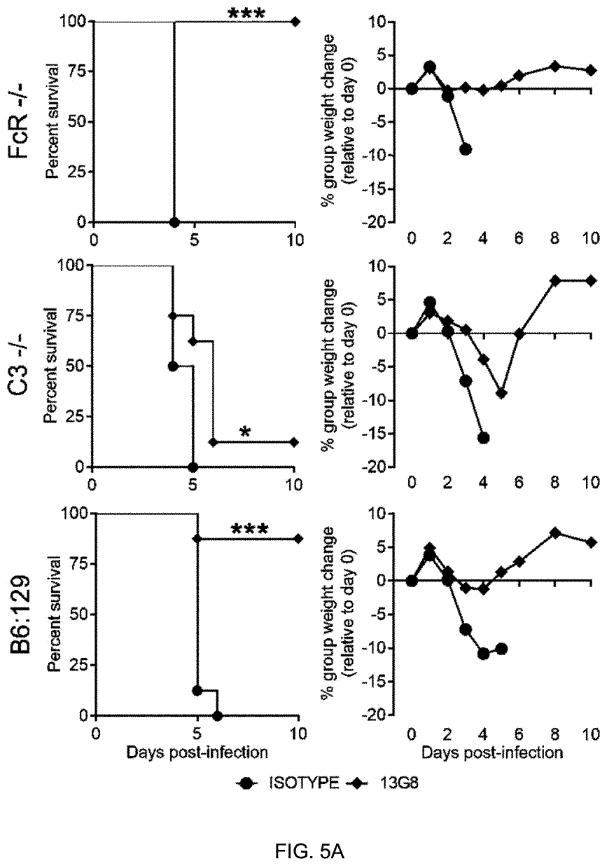

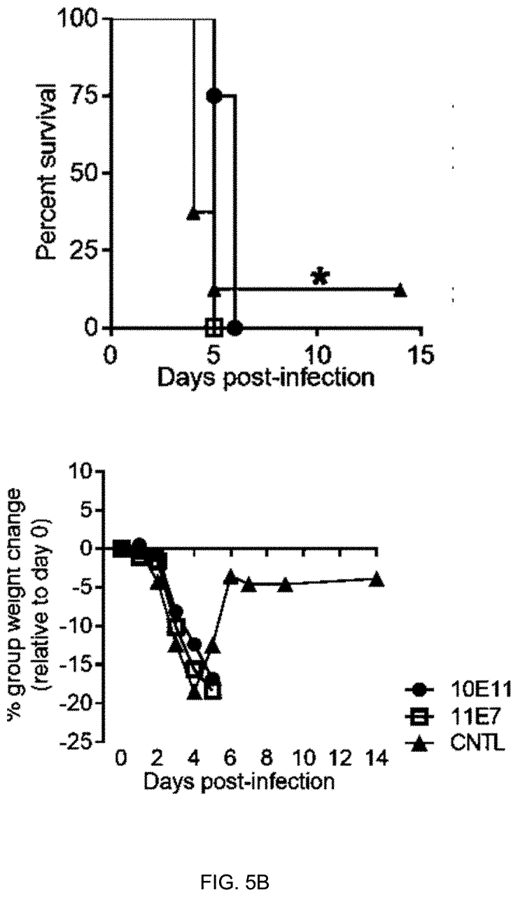

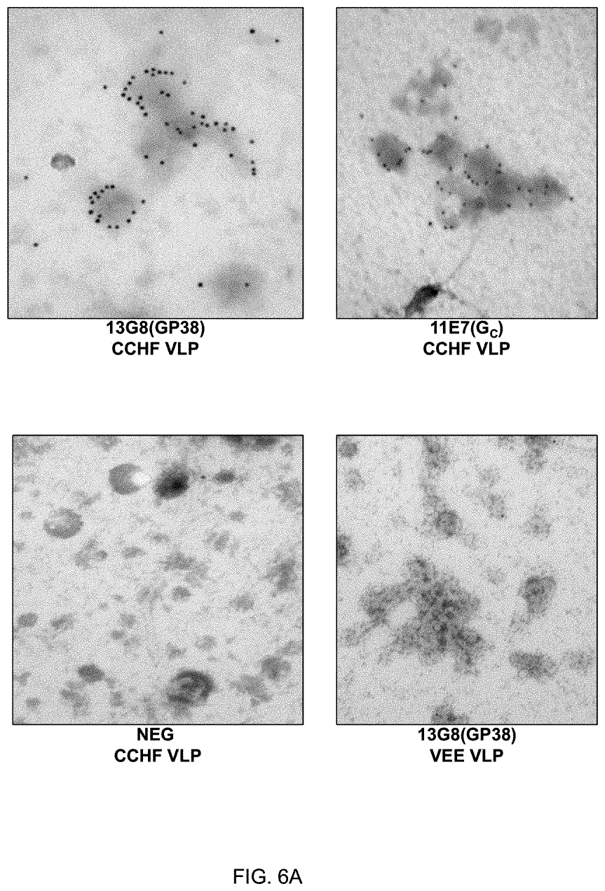

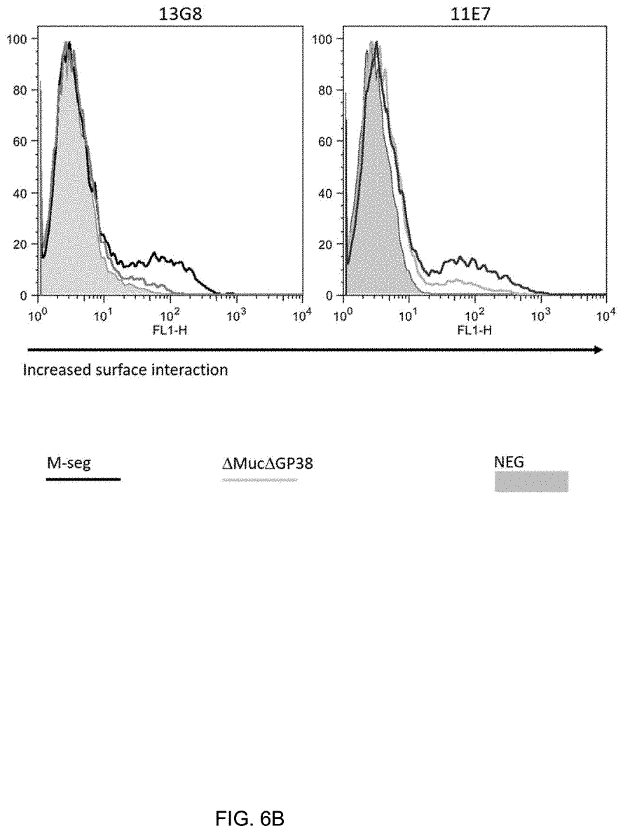

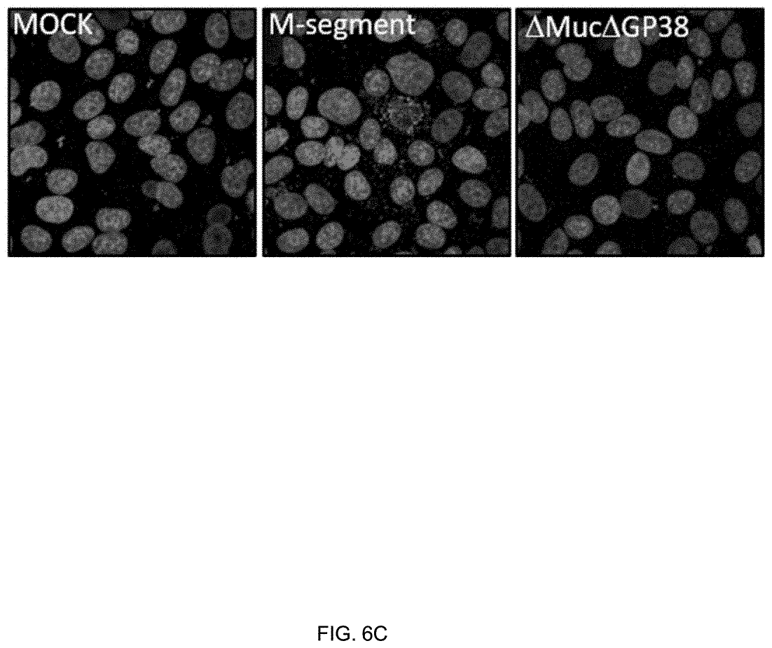

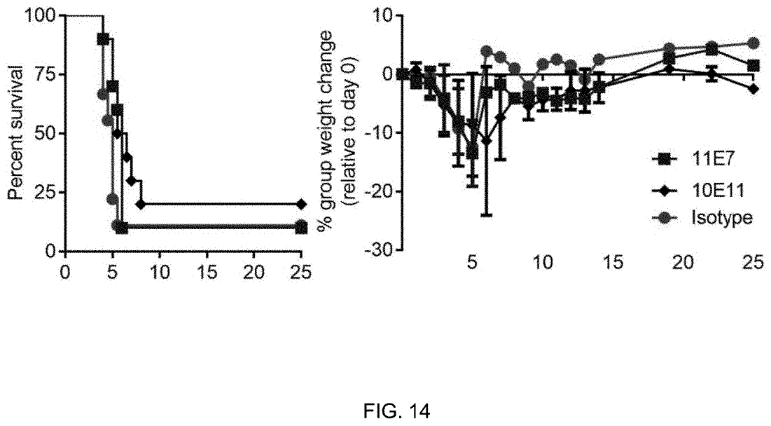

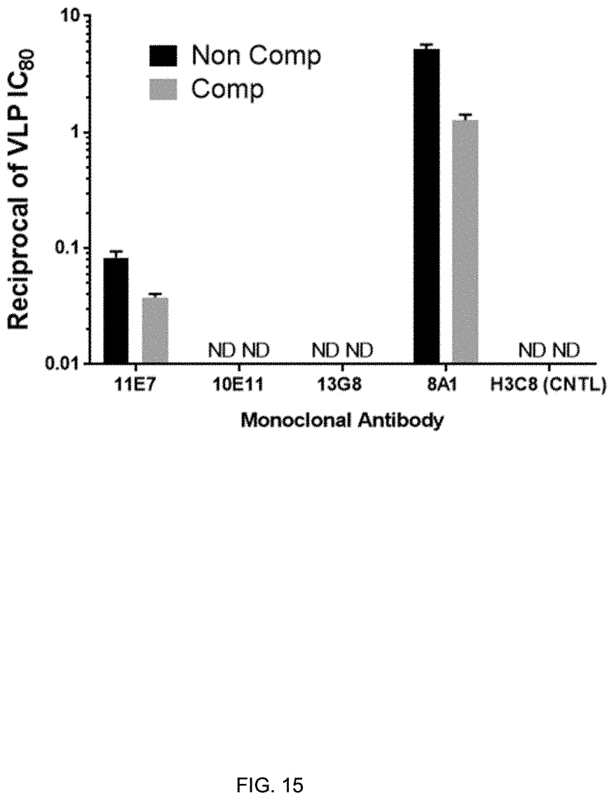

To determine if GP38 is present in viral envelope, CCHF virus like particles (VLPs) were stained with mAb-13G8 (anti-GP38) or mAb-11E7 (anti-G_{C}) or an irrelevant antibody. Irrelevant VLPs, derived from Venezuelan equine encephalitis virus (VEEV) surface proteins were also stained, with the mAb-13G8. Particles were then stained with immuno-gold labeled secondary antibodies and examined by electron microscopy. CCHF, but not VEE, VLPs were positive for both GP38 and G_{C} as indicated by mAb-13G8 staining respectively (FIG. 6A). This suggested that GP38 can localize to virus envelope. To evaluate localization of GP38 to cellular plasma membranes, 293T cells were transfected with plasmids encoding the full length M-segment, ΔMucΔGP38, or a negative control and the surface expression of GP38 and G_{C} was quantitated by flow cytometry using mAb-13G8 or mAb-11E7 under non-permeabilized conditions. GP38 was detected in the full-length M-segment expressing cells, but no signal was detected in the ΔMucΔGP38 or negative control cells. G_{C} could be detected in both the full-length M-segment and the ΔMucΔGP38 expressing cells, but to a slightly lesser amount in the latter (FIG. 6B). Using confocal microscopy, GP38 could also be detected on the surfaces of VeroE6 cells expressing M-segment but not mock transfected cells or cells expressing ΔMucΔGP38 (FIG. 6C). These data support a conclusion that in addition to being secreted, GP38 can localize to viral and cellular membranes. Despite the presence of GP38 on the VLP surface, mAb-13G8 did not neutralize CCHFV VLPs in the presence of complement (FIG. 15).

### Example 8. GP38 is a Target of Anti-CCHFV Antibody Responses in Humans

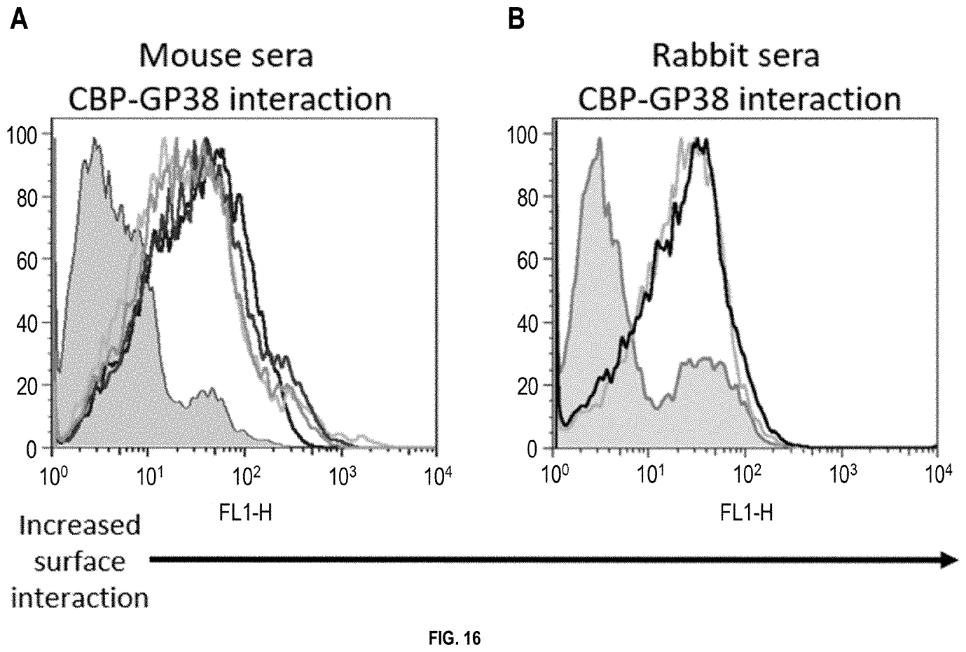

The panel of murine mAbs previously described to bind pre-G_{N} (20) were also evaluated for their ability to bind GP38 using a 6-His tagged GP38 molecule based on the IbAr10200 (GP38his). In addition to mAb-10E11 and mAb-13G8, all of the mAbs reported to bind pre-G_{N} in the Bertolotti-Ciarlet et al. paper interacted with GP38his (FIG. 7A). Furthermore, DNA vaccination of rabbits and mice with a plasmid encoding the CCHFV IbAr10200 M-segment elicited anti-GP38 antibodies in both species (FIG. 16). Because these data indicated GP38 to be a target of humoral immune responses in CCHFV infected and vaccinated animals, it was investigated if GP38-targeting antibodies were produced in infected humans. Convalescent human sera from two CCHFV survivors of African origin was evaluated for the presence of antibodies against G_{N}, GP38, and N protein by ELISA using purified proteins (FIG. 7B). Both CCHFV-infected humans produced antibody response against all three targets, whereas human sera from an unexposed human was negative for these same molecules. These findings indicate that GP38 is a natural immune target of antibody responses in CCHFV infected humans and vaccinated animals. Next, a tandem competition using Octet analysis assay was conducted to identify putative epitope binding groups based on the ability of members of this murine mAb library to compete against each other for GP38 binding (FIG. 7C). Recombinant GP38 was used as the antigen and the antibodies in FIG. 7A were interrogated, along with two non-specific control antibodies, mAb-11E7 (anti-CCHFV G_{C}) and mAb-H3C8 (Ebola virus GP). As expected, all antibodies competed efficiently against themselves (permissivity ≤25) and the non-specific control mAbs failed to block binding of any of the GP38-targeting mAbs to the GP38 antigen (permissivity ≥75). With regard to the GP38-targeting mAbs, this binding assay revealed three non-overlapping antibody competition groups, suggesting that at least three independent epitopes are present on the GP38 molecule.

### Example 9. Binding of Antibody to CBD-GP38

For this experiment, a poxvirus protein was exploited in which one region of that molecule, termed the cell binding domain (CBD), can bind to mammalian cell surfaces. GP38 from CCHFV strain IbAr10200 was de novo synthesized in-frame with the C-terminal end of the CBD. The resultant gene construction (CBD-GP38) was transfected into 293T cells using Fugene6 along with a negative control plasmid derived from Junin virus GP1 (CBP-GP1) (44). After three days, cells were harvested, washed in FACS buffer [PBS +5% fetal bovine serum (FBS)]. Cells were incubated with the indicated antibodies at a concentration of 1:100 diluted in FACS buffer for 1 h at 37° C. Cells were washed three times in FACS buffer and pelleted via low speed centrifugation. Cells were then incubated with a species-specific secondary antibody conjugated to AlexaFluor488 (1:500) for 30 mat 37° C. Samples were then washed three times and resuspended in fresh FACS buffer and interaction of various antibodies evaluated by flow cytometry. Flow cytometry was performed on a FACSCalibur flow cytometer (Becton Dickinson). Data were collected and analyzed using FlowJo software (Tree Star INC; Ashland, Oreg.). A total of 10,000 cells were analyzed for each sample using a live-gate.

### Example 10. Vaccination of Mice Against CCHFV M-Segment

Mice were vaccinated as described in (45). C57BL/6 mice were vaccinated in the anterior tibialis muscle with 25 μg of either the CCHFV-M co-DNA vaccine (IbAr10200) using the Ichor TriGrid® IM-EP system, under isoflurane anesthesia. All mice were vaccinated three times at three weeks intervals. Blood was obtained via submandibular bleeds three weeks after the third vaccination.

### Example 11. CCHF VLP Production

CCHF VLPs production of IbAr10200 strain (CCHFVLP) was performed as reported previously (46). Briefly, BHK-21 cells were transfected with 10 μg pC-M Opt (IbAr10200), 4 μg PC-N, 2 μg L-Opt, 4 μg T7-Opt, and 1 μg Nano-luciferase encoding mini-genome plasmid using the Transit LT-1 transfection reagent according to manufacturer's instructions (Mirus Bio). Supernatants were harvested on 3 d, clarified by low speed centrifugation. Subsequently, VLPs were pelleted through a cushion of 20% sucrose in virus resuspension buffer (VRB; 130 mM NaCl, 20 mM HEPES, pH 7.4) by centrifugation for 2 h at 106,750× g in an SW32 rotor at 4° C. and then in 1/200 volume VRB at 4° C. VLPs were frozen at −80° C. in single-use aliquots. Individual lots of CCHFVLP were standardized by Western Blot analysis based on incorporation of NP relative to a parallel gradient of recombinant NP loaded on the same SDS-PAGE reducing gel. CCHFVLP were quantified using a TCID50 assay on SW13 cells in 96-well, black-walled, clear-bottom plates (Costar). Plates were incubated with tenfold dilutions of the CCHFVLP overnight and were then processed for Nano Luciferase (Promega) expression. Wells that displayed a Nano Luciferase signal 3 standard deviations or greater above background levels were considered positive for VLP signal. VLP stock concentrations (TCID50 per mL) were calculated using the Reed and Muench formula (47).

### Example 12. CCHFV VLP Neutralization Assay

VLP neutralization was performed as described previously (45). Briefly, 24 h prior to use, 50,000 SW13 cells were seeded into a 96 well black-walled tissue culture plate. The indicated antibodies were half-log serially diluted (from 1:25 to 1:25,368) and then an equal volume of medium with IbAr10200 VLPs containing 237 TCID50 units was added and incubated at 37° C./5% CO2 for 1 h. For some samples, 5% Low-Tox Guinea Pig Complement was included in the dilution (Cederlane labs). Half of this reaction mixture (50 μl) was then added to the previously aspirated target cell plate. Cells were incubated for 24 h before being lysed using NanoGlo Lysis buffer mixed with 1/50 dilution of NanoGlo substrate (Promega). Samples were mixed and incubated for 5 min at ambient temperature prior to the luminescent signal being measured on a Modulus Microplate Reader (Turner Biosystems) with an integration time of 5 s per well. VLP neutralization (80% inhibition) was generated for each mAb as previously described (Aura 2017 ref) both with and without 5% rodent complement. The reciprocal value of the mAb concentration corresponding to the VLPNeut80 was then calculated (e.g. mAb with a VLPNeut80 of 1 μg/ml would be reported as 1, 10 μg/ml as 0.1, etc. Data were analyzed as previously reported using GraphPad Prism software (48).

### Example 13. VLP Octet Interactions

A premix competition assays was used for binning the three CCHF antibodies (11E7, 10E11, 13G8). The premix competition assay consisted of four parts. Part one was to bind the stationary antibody onto a sensor. Part two was to premix the saturating antibody with biotinylated VLPs and incubate. Part three was to interrogate the sensors with the premix solution. Part four was to obtain a baseline response followed by response of the sensors in the amplification wells.

### Part One: Preparation of Sensors

The Amine Reactive Second Generation (AR2G) (Pall Corp Forte Bio) sensors were loaded with monoclonal antibody using following steps. 1. Water wash. (60 seconds) 2. ED+NHS activation. (300 seconds) 3. Loading antibody. (300 seconds) 4. Quenching with ethanolamine. (300 seconds) 5. Equilibration in Kinetics Buffer. (60 seconds) All of the steps were performed at 1000 RPM and the temperature set to 30 Celsius on the Octet QKe. Sensors were then held in Kinetics Buffer until premixed VLPs were ready for interrogation.

### Part Two: Preparation of Premix

CCHF VLPs at a concentration of 10 μg/mL were mixed with a saturating antibody at 20 μg/mL concentration for 20 minutes using the Octet QKe. A null antibody (anti-EBOV GP-H3C8) was also included and a well with no antibody as a negative control well. The incubation step was performed at 30 Celsius with a shaking rate of 1000 RPM.

### Part three: Interrogate sensors with premix solutions.

All of the following steps were performed at 300 RPM and 30C on the Octet QKe. The sensors were blocked for 3 minutes using a solution of Kinetics buffer with 0.1% goat serum (SeraCare). The sensors were then washed for 60 seconds in well of Kinetics Buffer. The sensors were then moved to the premixed solutions of VLPs with antibodies or controls. The premix and sensors were incubated together for 30 minutes on the Octet, followed by another 3 minute block in Kinetics buffer with 0.1% Goat sera and a one minute wash in Kinetics Buffer.

### Part Four: Baseline and Signal Amplification

All of the following steps were performed at 300 RPM and 30° C. on the Octet QKe. The sensors were incubated in wells containing Streptavidin HRP Sigma) 0.05% in Kinetics Buffer for five minutes. Sensors were then moved to wells with kinetics buffer for two minutes. The binding average from last ten seconds of the kinetics step was used as the baseline for the following step. The sensors were then placed in DAB solution (ThermoFisher). The DAB solution makes a precipitate on the sensor surface in the presence of HRP. The precipitate amplifies the binding signal, which indicates any binding activity that occurred during the interrogation step.

While specific aspects of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

### REFERENCES

1. C. A. Whitehouse, Crimean-Congo hemorrhagic fever. Antiviral Res 64, 145-160 (2004).
2. O. Ergonul, Crimean-Congo haemorrhagic fever. Lancet Infect Dis 6, 203-214 (2006).
3. D. A. Bente et al., Crimean-Congo hemorrhagic fever: history, epidemiology, pathogenesis, clinical syndrome and genetic diversity. Antiviral Res 100, 159-189 (2013).
4. A. J. Shepherd, P. A. Leman, R. Swanepoel, Viremia and antibody response of small African and laboratory animals to Crimean-Congo hemorrhagic fever virus infection. Am J Trop Med Hyg 40, 541-547 (1989).
5. J. R. Spengler et al., A chronological review of experimental infection studies of the role of wild animals and livestock in the maintenance and transmission of Crimean-Congo hemorrhagic fever virus. Antiviral Res 135, 31-47 (2016).
6. A. Negredo et al., Autochthonous Crimean-Congo Hemorrhagic Fever in Spain. N Engl J Med 377, 154-161 (2017).
7. N. G. Conger et al., Health care response to CCHF in US soldier and nosocomial transmission to health care providers, Germany, 2009. Emerg Infect Dis 21, 23-31 (2015).
8. O. Ergonul et al., Characteristics of patients with Crimean-Congo hemorrhagic fever in a recent outbreak in Turkey and impact of oral ribavirin therapy. Clin Infect Dis 39, 284-287 (2004).
9. I. Koksal et al., The efficacy of ribavirin in the treatment of Crimean-Congo hemorrhagic fever in Eastern Black Sea region in Turkey. J Clin Virol 47, 65-68 (2010).
10. A. Hiatt et al., The emergence of antibody therapies for Ebola. Hum Antibodies 23, 49-56 (2015).
11. J. I. Maiztegui, N. J. Fernandez, A. J. de Damilano, Efficacy of immune plasma in treatment of Argentine haemorrhagic fever and association between treatment and a late neurological syndrome. Lancet 2, 1216-1217 (1979).
12. M. Keshtkar-Jahromi et al., Crimean-Congo hemorrhagic fever: current and future prospects of vaccines and therapies. Antiviral Res 90, 85-92 (2011).
13. M. N. Suleiman et al., Congo/Crimean haemorrhagic fever in Dubai. An outbreak at the Rashid Hospital. Lancet 2, 939-941 (1980).
14. S. M. Vassilenko, T. L. Vassilev, L. G. Bozadjiev, I. L. Bineva, G. Z. Kazarov, Specific intravenous immunoglobulin for Crimean-Congo haemorrhagic fever. Lancet 335, 791-792 (1990).
15. E. Bergeron, M. J. Vincent, S. T. Nichol, Crimean-Congo hemorrhagic fever virus glycoprotein processing by the endoprotease SKI-1/S1P is critical for virus infectivity. Journal of Virology 81, 13271-13276 (2007).
16. A. J. Sanchez, M. J. Vincent, S. T. Nichol, Characterization of the glycoproteins of Crimean-Congo hemorrhagic fever virus. J Virol 76, 7263-7275 (2002).
17. M. Zivcec, F. E. Scholte, C. F. Spiropoulou, J. R. Spengler, E. Bergeron, Molecular Insights into Crimean-Congo Hemorrhagic Fever Virus. Viruses 8, 106 (2016).
18. L. A. Altamura et al., Identification of a novel C-terminal cleavage of Crimean-Congo hemorrhagic fever virus PreG(N) that leads to generation of an NSM protein. Journal of Virology 81, 6632-6642 (2007).
19. A. J. Sanchez, M. J. Vincent, B. R. Erickson, S. T. Nichol, Crimean-congo hemorrhagic fever virus glycoprotein precursor is cleaved by Furin-like and SKI-1 proteases to generate a novel 38-kilodalton glycoprotein. J Virol 80, 514-525 (2006).
20. A. Bertolotti-Ciarlet et al., Cellular localization and antigenic characterization of crimean-congo hemorrhagic fever virus glycoproteins. J Virol 79, 6152-6161 (2005).
21. M. E. Lindquist et al., Exploring Crimean-Congo hemorrhagic fever virus-induced hepatic injury using antibody-mediated type I interferon blockade in mice. J Virol, (2018).
22. M. Zivcec et al., Lethal Crimean-Congo hemorrhagic fever virus infection in interferon alpha/beta receptor knockout mice is associated with high viral loads, proinflammatory responses, and coagulopathy. J Infect Dis 207, 1909-1921 (2013).
23. S. Bereczky et al., Crimean-Congo hemorrhagic fever virus infection is lethal for adult type I interferon receptor-knockout mice. J Gen Virol 91, 1473-1477 (2010).
24. A. L. Schmaljohn, Protective antiviral antibodies that lack neutralizing activity: precedents and evolution of concepts. Curr HIV Res 11, 345-353 (2013).
25. E. A. Henchal, L. S. Henchal, J. J. Schlesinger, Synergistic interactions of anti-NS1 monoclonal antibodies protect passively immunized mice from lethal challenge with dengue 2 virus. J Gen Virol 69 (Pt 8), 2101-2107 (1988).
26. J. A. Horwitz et al., Non-neutralizing Antibodies Alter the Course of HIV-1 Infection In Vivo. Cell 170, 637-648 e610 (2017).
27. A. L. Schmaljohn, E. D. Johnson, J. M. Dalrymple, G. A. Cole, Non-neutralizing monoclonal antibodies can prevent lethal alphavirus encephalitis. Nature 297, 70-72 (1982).
28. K. M. Chung et al., Antibodies against West Nile Virus nonstructural protein NS1 prevent lethal infection through Fc gamma receptor-dependent and -independent mechanisms. J Virol 80, 1340-1351 (2006).
29. A. R. Garrison et al., A DNA vaccine for Crimean-Congo hemorrhagic fever protects against disease and death in two lethal mouse models. PLoS Negl Trop Dis 11, e0005908 (2017).
30. X. Xiao, Y. Feng, Z. Zhu, D. S. Dimitrov, Identification of a putative Crimean-Congo hemorrhagic fever virus entry factor. Biochem Biophys Res Commun 411, 253-258 (2011).
31. H. Wilde, P. Chomchey, P. Punyaratabandhu, P. Phanupak, S. Chutivongse, Purified equine rabies immune globulin: a safe and affordable alternative to human rabies immune globulin. Bull World Health Organ 67, 731-736 (1989).
32. K. Mupapa et al., Treatment of Ebola hemorrhagic fever with blood transfusions from convalescent patients. International Scientific and Technical Committee. J Infect Dis 179 Suppl 1, S18-23 (1999).
33. S. D. Dowall et al., A Crimean-Congo Haemorrhagic Fever (CCHF) viral vaccine expressing nucleoprotein is immunogenic but fails to confer protection against lethal disease. Hum Vaccin Immunother, 0 (2015).
34. M. Rastogi, N. Sharma, S. K. Singh, Flavivirus NS1: a multifaceted enigmatic viral protein. Virol J 13, 131 (2016).
35.M. Zivcec et al., Identification of broadly neutralizing monoclonal antibodies against Crimean-Congo hemorrhagic fever virus. Antiviral Res 146, 112-120 (2017).
36. J. Hinkula et al., Immunization with DNA Plasmids Coding for Crimean-Congo Hemorrhagic Fever Virus Capsid and Envelope Proteins and/or Virus-Like Particles Induces Protection and Survival in Challenged Mice. J Virol, (2017).
37. Z. Feng et al., A pathogenic picornavirus acquires an envelope by hijacking cellular membranes. Nature 496, 367-371 (2013).
38. Z. Feng, S. M. Lemon, Peek-a-boo: membrane hijacking and the pathogenesis of viral hepatitis. Trends Microbiol 22, 59-64 (2014).
39. A. Estrada-Pena et al., Crimean-Congo hemorrhagic fever virus in ticks, Southwestern Europe, 2010. Emerg Infect Dis 18, 179-180 (2012).

### 40. N. R. Council, Guide for the Care and Use of Laboratory Animals. (National Academies Press, Washington, D.C., ed. 8th, 2011).

41. S. Olschlager et al., Complete sequence and phylogenetic characterisation of Crimean-Congo hemorrhagic fever virus from Afghanistan. J Clin Virol 50, 90-92 (2011).
42. E. B. Prophet, Mills, B., Arrington, J. B., Sobin, L. H., Laboratory methods for histotechnology. Armed Forces Institute of Pathology, Washington, D.C. , (1992).
43. J. W. Golden, P. Maes, S. A. Kwilas, J. Ballantyne, J. W. Hooper, Glycoprotein-Specific Antibodies Produced by DNA Vaccination Protect Guinea Pigs from Lethal Argentine and Venezuelan Hemorrhagic Fever. J Virol 90, 3515-3529 (2016).
44. J. W. Golden, P. Maes, S. A. Kwilas, J. Ballantyne, J. W. Hooper, Glycoprotein-Specific Antibodies Produced by DNA Vaccination Protect Guinea Pigs from Lethal Argentine and Venezuelan Hemorrhagic Fever. J Virol 90, 3515-3529 (2016).
45. A. R. Garrison et al., A DNA vaccine for Crimean-Congo hemorrhagic fever protects against disease and death in two lethal mouse models. PLoS Negl Trop Dis 11, e0005908 (2017).
46. M. Zivcec et al., Identification of broadly neutralizing monoclonal antibodies against Crimean-Congo hemorrhagic fever virus. Antiviral Res 146, 112-120 (2017).
47. B. D. Lindenbach, Measuring HCV infectivity produced in cell culture and in vivo. Methods Mol Biol 510, 329-336 (2009).
48. S. Kwilas et al., A hantavirus pulmonary syndrome (HPS) DNA vaccine delivered using a spring-powered jet injector elicits a potent neutralizing antibody response in rabbits and nonhuman primates. Curr Gene Ther 14, 200-210 (2014).

1. A non-neutralizing antibody for treatment against CCHFV infection, wherein the non-neutralizing antibody binds specifically to GP38.

2. The antibody of claim 1, wherein the antibody binds specifically to the amino acid sequence set forth in SEQ ID NO:1, or variants or fragments thereof.

3. The antibody of claim 1, wherein the antibody comprises heavy chain CDR1, CDR2, and CDR3 having the same amino acid sequences as heavy chain CDR1, CDR2, and CDR3 of antibody mAb-13G8 and light chain CDR1, CDR2, and CDR3 having the same amino acid sequences as light chain CDR1, CDR2, and CDR3 of antibody mAb-13G8.

4. A fragment of the antibody of claim 1, which has specific binding activity to GP38, or variants or fragments thereof.

5. A chimeric or a humanized antibody of claim 1.

6. A method of treating or preventing CCHFV infection in a subject wherein the subject is administered a composition comprising the antibody of claim 1.

7. The method of claim 6, wherein the antibody comprises heavy chain CDR1, CDR2, and CDR3 having the same amino acid sequences as heavy chain CDR1, CDR2, and CDR3 of antibody mAb-13G8 and light chain CDR1, CDR2, and CDR3 having the same amino acid sequences as light chain CDR1, CDR2, and CDR3 of antibody mAb-13G8.

8. The method of claim 6, wherein the antibody is administered to a subject after infection by CCHFV.

9. The method of claim 6, wherein the antibody is administered to a subject at risk of exposure to CCHFV.

10. A method for producing a chimeric antibody or humanized antibody of the antibody of claim 1, which comprises linking a DNA encoding a variable region of the antibody of claim 1 with a DNA encoding a constant region; inserting this into an expression vector; introducing the vector into a host; and
producing the variable region and the constant region of the antibody.

11. The method of claim 10, wherein the antibody comprises heavy chain CDR1, CDR2, and CDR3 having the same amino acid sequences as heavy chain CDR1, CDR2, and CDR3 of antibody mAb-13G8 and light chain CDR1, CDR2, and CDR3 having the same amino acid sequences as light chain CDR1, CDR2, and CDR3 of antibody mAb-13G8.

12. The method of claim 6, wherein the subject is a mammal.

13. A humanized antibody for treating a CCHFV infection in a mammalian subject, wherein the antibody specifically binds to the amino acid sequence set forth in SEQ ID NO:1, or an amino acid sequence having at least 80% sequence identity to SEQ ID NO:1.

14. The humanized antibody of claim 13 that comprises a DNA encoding a variable region of the antibody and a DNA encoding a constant region derived from a human antibody.

15. The humanized antibody of claim 14, wherein the antibody comprises heavy chain CDR1, CDR2, and CDR3 having the same amino acid sequences as heavy chain CDR1, CDR2, and CDR3 of antibody mAb-13G8 and light chain CDR1, CDR2, and CDR3 having the same amino acid sequences as light chain CDR1, CDR2, and CDR3 of antibody mAb-13G8.

16. The humanized antibody of claim 15, wherein at least three independent epitopes are present and associated with SEQ ID NO:1.
